(19) United States
(12) Patent Application Publication
(10) Pub. No.: US 2022/0005556 A1
(43) Pub. Date: Jan. 6, 2022

(54) METHOD AND APPARATUS FOR MANAGING CLINICAL TRIALS AND RESEARCH

(71) Applicant: Devana Solutions, LLC., Mt. Pleasant, SC (US)

(72) Inventors: Barry S. LAKE, Chapel Hill, NC (US); Sean P. RICE, Mt. Pleasant, SC (US)

(21) Appl. No.: 17/391,827

(22) Filed: Aug. 2, 2021

Related U.S. Application Data
(63) Continuation of application No. 15/765,440, filed on Apr. 2, 2018.
(60) Provisional application No. 62/238,860, filed on Oct. 8, 2015.

(51) Int. Cl. G16H 10/20; G06F 16/23; G16H 40/67; G06F 3/0484; G06Q 10/06; G06Q 10/10

(52) U.S. Cl. CPC G16H 10/20; G06F 16/23; G16H 40/67; G06N 10/00; G06Q 10/0639; G06Q 10/1091; G06F 3/0484

(57) ABSTRACT

An approach is provided for managing clinical trials and research. The approach involves receiving, by a clinical trial management platform, an input via a user interface element of a user interface of the clinical trial management platform. The input specifies an edit or an update to a data record associated with tracking a clinical trial process. The approach also involves determining a workflow rule associated with the user interface element, which specifies criteria for initiating a capture of a timing metric and/or a performance metric associated with the clinical trial process. The approach further involves initiating the capture of the timing metric and/or the performance metric based on an evaluation of the input against the one or more criteria to update the data record. The approach further involves updating a plurality of user interface elements that are visible in the user interface based on the updated data record.

### RELATED APPLICATIONS

This application is a continuation of U.S. application Ser. No. 15/765,440, entitled “METHOD AND APPARATUS FOR MANAGING CLINICAL TRIALS AND RESEARCH”, filed Apr. 2, 2018, which claims priority from PCT Application Serial No. PCT/US2016/56059, entitled “METHOD AND APPARATUS FOR MANAGING CLINICAL TRIALS AND RESEARCH,” filed Oct. 7, 2016, which claims priority from U.S. Provisional Patent Application No. 62/238,860, titled “Method and Apparatus for Managing Clinical Trials and Research,” filed Oct. 8, 2015, the entire disclosure of which is hereby incorporated by reference herein.

### BACKGROUND

Historically, designing, implementing, and managing clinical trials has been a complex management challenge. For example, clinical trials and research often span over significant periods of time (e.g., years) and involve participation by multiple and potentially differing teams of participants at various stages of the trial process. In many cases, these stages are associated with specific workflows that set out, for instance, goals and/or tasks to achieve those goals. As a result, there is a need for an approach for a service for identifying potential trials to conduct as well as for coordinating those clinical trials and/or research studies that users (e.g., clinical research sites) select to conduct.

### SOME EXAMPLE EMBODIMENTS

Therefore, there is a need for an automated platform to assist users (e.g., clinical research sites) in managing the complex information management and analysis functions associated with competing for and/or conducting studies in support of clinical trials or other similar processes.

According to one embodiment, a method comprises receiving, by a clinical trial management platform, an input via a user interface element of a user interface of the clinical trial management platform. The input specifies an edit or an update to a data record associated with tracking a clinical trial process. The method also comprises determining a workflow rule associated with the user interface element. The workflow rule specifies one or more criteria for initiating a capture of a timing metric, a performance metric, or a combination thereof associated with the clinical trial process. The method further comprises initiating the capture of the timing metric, the performance metric, or a combination thereof based on an evaluation of the input against the one or more criteria. The method further comprises storing the timing metric, the performance metric, or a combination thereof to update the data record. The method further comprises updating a plurality of user interface elements that are visible in the user interface based on the updated data record.

According to another embodiment, an apparatus comprises at least one processor, and at least one memory including computer program code for one or more computer programs, the at least one memory and the computer program code configured to, with the at least one processor, cause, at least in part, the apparatus to receive, by a clinical trial management platform, an input via a user interface element of a user interface of the clinical trial management platform. The input specifies an edit or an update to a data record associated with tracking a clinical trial process. The apparatus is also caused to determine a workflow rule associated with the user interface element. The workflow rule specifies one or more criteria for initiating a capture of a timing metric, a performance metric, or a combination thereof associated with the clinical trial process. The apparatus is further caused to initiate the capture of the timing metric, the performance metric, or a combination thereof based on an evaluation of the input against the one or more criteria. The apparatus is further caused to store the timing metric, the performance metric, or a combination thereof to update the data record. The apparatus is further caused to update a plurality of user interface elements that are visible in the user interface based on the updated data record.

According to another embodiment, a computer-readable storage medium carries one or more sequences of one or more instructions which, when executed by one or more processors, cause, at least in part, an apparatus to receive, by a clinical trial management platform, an input via a user interface element of a user interface of the clinical trial management platform. The input specifies an edit or an update to a data record associated with tracking a clinical trial process. The apparatus is also caused to determine a workflow rule associated with the user interface element. The workflow rule specifies one or more criteria for initiating a capture of a timing metric, a performance metric, or a combination thereof associated with the clinical trial process. The apparatus is further caused to initiate the capture of the timing metric, the performance metric, or a combination thereof based on an evaluation of the input against the one or more criteria. The apparatus is further caused to store the timing metric, the performance metric, or a combination thereof to update the data record. The apparatus is further caused to update a plurality of user interface elements that are visible in the user interface based on the updated data record.

According to another embodiment, an apparatus comprises means for receiving, by a clinical trial management platform, an input via a user interface element of a user interface of the clinical trial management platform. The input specifies an edit or an update to a data record associated with tracking a clinical trial process. The apparatus also comprises means for determining a workflow rule associated with the user interface element. The workflow rule specifies one or more criteria for initiating a capture of a timing metric, a performance metric, or a combination thereof associated with the clinical trial process. The apparatus further comprises means for initiating the capture of the timing metric, the performance metric, or a combination thereof based on an evaluation of the input against the one or more criteria. The apparatus further comprises means for storing the timing metric, the performance metric, or a combination thereof to update the data record. The apparatus further comprises means for updating a plurality of user interface elements that are visible in the user interface based on the updated data record.

In addition, for various example embodiments of the invention, the following is applicable: a method comprising facilitating a processing of and/or processing (1) data and/or (2) information and/or (3) at least one signal, the (1) data and/or (2) information and/or (3) at least one signal based, at least in part, on (or derived at least in part from) any one or any combination of methods (or processes) disclosed in this application as relevant to any embodiment of the invention.

For various example embodiments of the invention, the following is also applicable: a method comprising facilitating access to at least one interface configured to allow access to at least one service, the at least one service configured to perform any one or any combination of network or service provider methods (or processes) disclosed in this application.

For various example embodiments of the invention, the following is also applicable: a method comprising facilitating creating and/or facilitating modifying (1) at least one device user interface element and/or (2) at least one device user interface functionality, the (1) at least one device user interface element and/or (2) at least one device user interface functionality based, at least in part, on data and/or information resulting from one or any combination of methods or processes disclosed in this application as relevant to any embodiment of the invention, and/or at least one signal resulting from one or any combination of methods (or processes) disclosed in this application as relevant to any embodiment of the invention.

For various example embodiments of the invention, the following is also applicable: a method comprising creating and/or modifying (1) at least one device user interface element and/or (2) at least one device user interface functionality, the (1) at least one device user interface element and/or (2) at least one device user interface functionality based at least in part on data and/or information resulting from one or any combination of methods (or processes) disclosed in this application as relevant to any embodiment of the invention, and/or at least one signal resulting from one or any combination of methods (or processes) disclosed in this application as relevant to any embodiment of the invention.

In various example embodiments, the methods (or processes) can be accomplished on the service provider side or on the mobile device side or in any shared way between service provider and mobile device with actions being performed on both sides.

For various example embodiments, the following is applicable: An apparatus comprising means for performing the method of any the claims.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The embodiments of the invention are illustrated by way of example, and not by way of limitation, in the figures of the accompanying drawings:

FIG. 1 is a diagram of a system capable of managing various stages of clinical trials and/or research studies, according to one example embodiment;

FIG. 2A is a flowchart of a process for generating a user interface for capturing time and performance metrics of a clinical trial process based on workflow rules, according to one embodiment;

FIGS. 2B and 2C are diagrams illustrating example notification templates, according to various embodiments;

FIG. 3A is a diagram that represents available contact record types, according to one example embodiment;

FIG. 3B is a diagram that represents available account record types, according to one example embodiment;

FIG. 4A is a diagram that represents the available trial record types for a single site use case, according to one example embodiment;

FIG. 4B is a diagram that represents the available trial record types for a multiple site use case, according to one example embodiment;

FIG. 5 is a diagram that represents Contract Research Organizations (CRO) rollups, according to one example embodiment;

FIG. 6 is a diagram that represents sponsor rollups, according to one example embodiment;

FIG. 7 is a diagram that represents indication rollups, according to one example embodiment;

FIG. 8 is a diagram that represents enrollment update rollups, according to one example embodiment;

FIG. 9 is a diagram that represents advertisement campaigns rollups, according to one example embodiment;

FIG. 10 is a flow chart that represents the relationship of all objects (both standard and custom), according to one example embodiment;

FIG. 11 is a diagram of a computer system that can be used to implement various exemplary embodiments; and

FIG. 12 is a diagram of a chip set that can be used to implement various exemplary embodiments.

### DESCRIPTION OF SOME EMBODIMENTS

Examples of a method, apparatus, and computer program for managing various stages of clinical trials and/or research studies are disclosed. In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention.

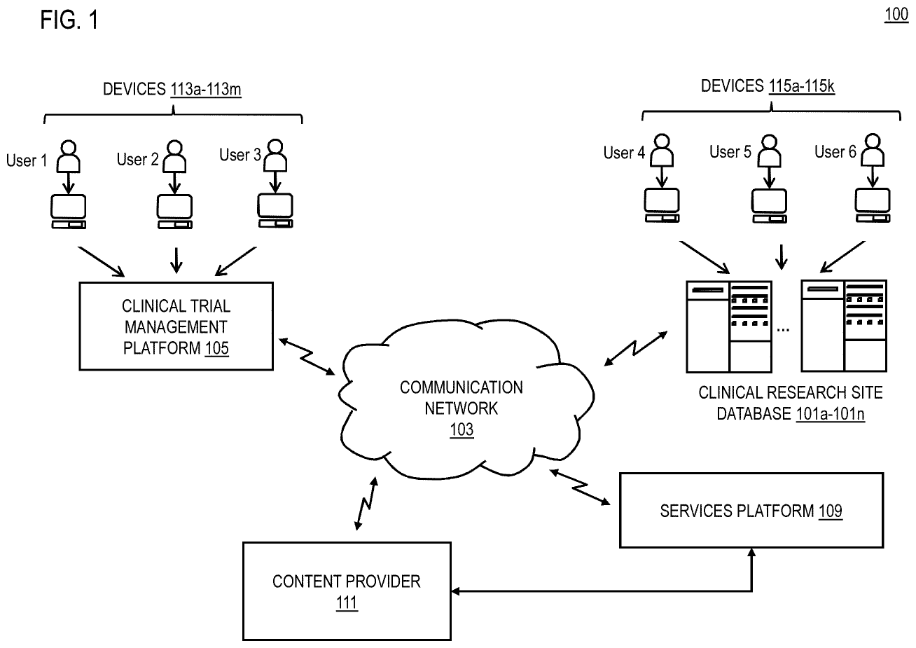

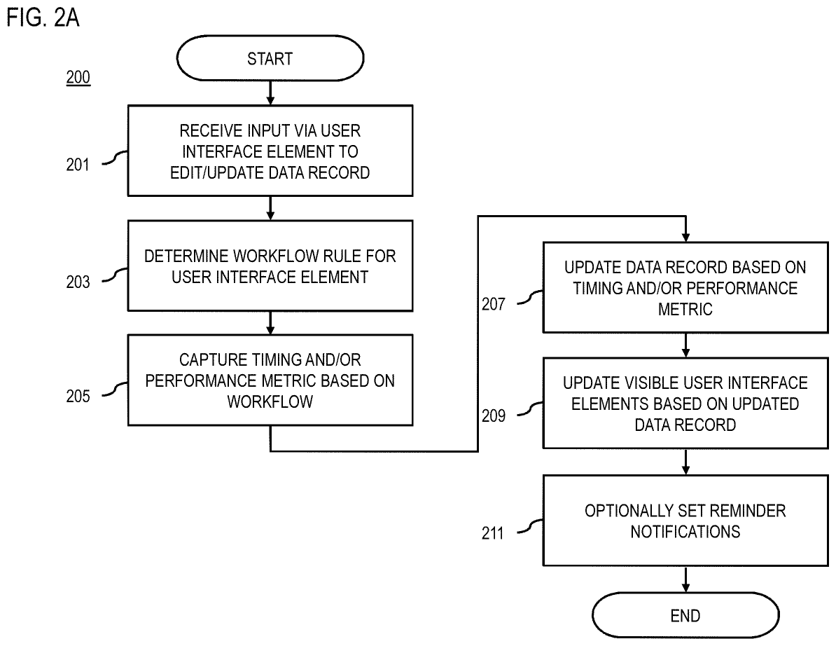

FIG. 1 is a diagram of a system capable of managing various stages of clinical trials and/or research studies, according to one example embodiment. As noted above, users and/or organizations who are seeking to conduct studies in support of clinical trials and/or who have decided to proceed with conducting a clinical trial face significant technical challenges in managing leads associated with new trials and resources/workflows associated with ongoing trials. In particular, the design, implementation and management of clinical research trials are extremely costly, complex and lengthy for a variety of reasons.

Firstly, a new drug can be in development for an inordinate length of time (e.g., 10-12 years) before it even reaches the clinical trials phase, the last step before a drug sponsor can seek FDA approval to bring the drug to market. In one example embodiment, in the U.S., an investigational new drug application (IND) needs to be submitted to the Food and Drug Administration (FDA) seeking permission to begin the clinical testing in human subjects. The clinical research (IND) phase representing the time from beginning of human trials to the new drug application (NDA) submission that seeks permission to market the drug can last up to 10 years.

Secondly, given the length of the sales cycle, a potential drug study or clinical trial can have multiple people or contacts at different levels performing different functions all related to the same Clinical Trial Opportunity. In one example embodiment, numerous researchers, lab-technicians, supervisors, etc. may perform various functions for the same clinical trial opportunity. Accordingly, there is a need for coordination of the various functions carried out by the multiple players to achieve the objective.

Thirdly, due to the intensely competitive nature of the industry where a blockbuster drug discovery can mean billions of dollars to the drug's sponsor/developer, confidentiality as a potential study moves through the clinical trials phase also adds to the complexity of attempting to track a new trial or study opportunity. In one scenario, disclosure and handling of drugs (e.g., patented or unpatented drugs) especially in the early research and development (R&D) stages raises the issue of confidentiality and related intellectual property protection. For this reason, it may be difficult to access and track privileged information pertaining to a new clinical trial.

Fourthly, the high degree of regulation governing the development and commercialization of pharmaceuticals adds complexity since research sites are required to be vetted to assess the ability of their staff and physician investigators to oversee and administer a very detailed protocol for dispensing and conducting experimental drug research on subject-patients in a safe environment. Drug development is highly regulated because of legitimate public health concerns.

Lastly, the time and complexity mentioned in the preceding four points, means that developing a new drug is an extremely costly proposition and, therefore, selecting of research sites to conduct a clinical drug trial is a multi-staged process that requires sites to submit feasibility studies and, undergo pre-study visits and screenings by sponsor and CRO (Contract Research Organization) personnel well before a research site is even considered for being formally awarded a chance to participate in a clinical trial.

For at least these above reasons, potential clinical trial research participants (e.g., research sites) face significant technical challenges to recording, tracking, and/or analyzing milestones, actions, and associated timing and performance metrics as they navigate the clinical trial process.

To address these challenges, a system 100 of FIG. 1 introduces, for instance, a service and/or application (e.g., cloud-based service/application) designed to enable users (e.g., clinical research sites) to manage their pipeline leads on future new clinical trials or research studies in, e.g., the pharmaceutical and/or medical device industries. In one embodiment, the system 100 provides technical functions that enable end users (e.g., research sites) manage trial studies or potential trial studies by: (1) automating study-lead tracking (e.g., via workflow rules), (2) managing contacts associated with study leads, (3) collecting timing and performance metrics associated with tracking study leads or conducting studies once a study as has been awarded, and (4) providing automated reminders of key events in the clinical trial process.

In one embodiment, to facilitate management of clinical trials, the system 100 provides a clinical trial management platform 105 using, for instance, a cloud-based service. In addition or alternatively, the clinical trial management platform 105 can be implemented in any other type of computing architecture such as a standalone application or service executed locally at client devices (e.g., devices 113a-113m—also collectively referred to as devices 113, or devices 115a-115k—also collectively referred to as devices 115). In one embodiment, the clinical trial management platform 105 can be developed as a managed package executing on programmable cloud-based customer relationship management (CRM) platform (e.g., CRM system provided by Salesforce.com, Inc.) or other similar system. For example, a managed package is a collection of application components that are posted as a unit into a hosted space in the CRM platform.

In one embodiment, the system 100 automatically captures a user's (e.g., a clinical research site or Contract Research Organization (CRO) timing and performance metrics as the user uses the clinical trial management platform 105 or other component of the system 100. In one embodiment, the clinical trial management platform 105 presents a user interface (e.g., a graphical user interface (GUI) to facilitate user interaction. As the user interacts with the GUI (e.g., to edit or update trial data records), the clinical trial management platform 105 may automatically date and time stamp key performance benchmarks or metrics to enable various teams (e.g., the executive leadership and business development teams) at clinical research sites to track and gain insight into their site's operating performance. In one embodiment, the system 100 may track and gain initial study lead through award letters, contract and budget negotiations, regulatory package submission, site initiation, patient enrollment, and so on.

These features enable users to gain insights that, for instance, can advantageously help the management teams at clinical research sites to correct deficiencies in any of the processes, and enables them to report their site's performance metrics. As a result, the clinical research sites may tout their capabilities at managing clinical trials to their drug company sponsor clients and CRO's and, ultimately, win more studies. In addition, the dynamic user interface of the clinical trial management platform 105 enables the user to more easily and quickly visualize clinical trial information by dynamically making stages or fields of a trial data record visible based on the current state or progress of the clinical trial process, thereby enable research site management to: (a) make decisions on improving in site performance in areas where the metrics indicate the site has inefficiencies; (b) make decisions to avoid certain types of trials where the site does not excel; (c) streamline progression from study lead to awarded clinical trial and through study completion (e.g., which can potentially span years); and (d) make decisions around scheduling of the site's work flow knowing certain CRO's are inefficient in managing study start-up procedures, etc.

It is noted that although the various embodiments discussed herein are described with respect to managing clinical trial processes, it is contemplated that the embodiments are also applicable to any industry. In other words, the embodiments of the system 100 described herein can provide any organization, regardless of industry, the ability to evaluate processes (e.g., by automatically collecting timing and performance metrics at process milestones).

In another embodiment, the system 100 comprises of numerous proprietary workflows that are governed by rules to fully automate the enormously complex process of tracking a potential research study from initial lead to final award letter and study start-up. In one embodiment, the system 100 creates and programs unique record types that can automatically determine what stages or fields of a data record are visible or applicable at particular states or milestones of the clinical trial process. In one embodiment, the system 100 can create data record types that relate to people or “Contacts”. Each record type may be customized to populate applicable fields pertaining to the selection. In another embodiment, the system 100 may create and program unique record types within “Accounts” based, at least in part, on the secrecy and competitive bid award process that takes place upstream in the sales cycle at the “Account” and/or drug company sponsor's requirement and/or the CRO's requirement. In a further embodiment, the system 100 may create and program eight unique record types within “Trials” based, at least in part, on the complexity of vetting, selecting, awarding and initiating the start-up of research sites during the “Trials” Phase.

As shown in FIG. 1, the system 100 comprises of clinical research site database 101a-101n (collectively referred to as database 101). In one embodiment, the database 101 has connectivity to a clinical trial management platform 105 via a communication network 103, e.g., a wireless communication network. In one embodiment, the clinical trial management platform 105 performs one or more functions associated with management of various stages of clinical trials and/or research studies.

In one embodiment, the database 101 may store and manage various content types for one or more clinical research sites. The information may be any multiple types of information that can provide means for aiding in the content provisioning and sharing process. In one example embodiment, the database 101 may include contact records, account records, trial records, or a combination thereof. In another example embodiment, the database 101 may include information on CRO rollups, sponsor rollups, indication rollups, enrollment update rollups, advertising campaign rollups, data flow information, or a combination thereof. By way of example, a rollup is a summary field of a record that calculates values from related records (e.g., records related through a master-detail relationship). In addition, rollups can be configured to perform various calculations using the related records including, but not limited, a sum, a minimum value, a maximum value, and the like.

In one embodiment, the system 100 may also provide a budgeting tool or user interface to support management of clinical trials or research via, e.g., the clinical trial management platform. The budgeting tool, for instance, can enable research site users to input budgeting information such as costs-per-procedure for performing clinical activities (e.g., physical examinations, tests—ECG or other tests, interpreting lab results, etc.) that may be conducted or required during a clinical trial. By way of example, the activities may be required and/or performed in accordance with protocols issued by study sponsors (e.g., pharmaceutical company sponsors) or their CRO.

In another embodiment, the budgeting tool enables research site users to associate their preferred amount (or range) of reimbursement or compensation for performing the clinical activities or procedures. The budgeting tool may also include a user interface or capability for researchers to enter what a sponsor or CRO's initial budget is offering to pay for each procedure. In addition or alternatively, other information indicating what the sponsor or CRO is offering to pay per procedure may be entered (e.g., a published pricing schedule, standard industry rates, etc.). In one embodiment, the system 100 (e.g., via the budgeting tool) can then calculate and analyze the desirability and/or acceptability of the budget offering against, e.g., the research site's compensation or other objectives (e.g., based on preconfigured algorithms, parameters, preferences, etc.).

In some embodiments, the budgeting tool may also analyze historical budget offering data that has been stored to calculate and/or generate a report for research site users. By way of example, the report may provide data including, but not limited to, the average amount offered per procedure historically versus what average amount per procedure that the research site has actually been paid during trials currently running or completed. This information can be used, for instance, by research sites in negotiating a final budget with the sponsor or CRO. In one embodiment, reporting may also look at budgeting data globally, by sponsor/CRO, by indication, by phase, and/or any other criteria. Ultimately, this budget feature, for instance, enables researchers to gauge the adequacy of the current budget begin proposed by a sponsor or their CRO and, will assist the site researchers and managers in their budget negotiations in order to manage a clinical trial in according with the research site's profitability objectives.

In one embodiment, the system 100 may also integrate or otherwise interface with one or more other clinical trial management systems (CTMS). In one embodiment, the integration enables research site users to manage “downstream” clinical activities (e.g., patient enrollment, patient visits, scheduling during clinical trial protocols, etc.) with the functions of the system 100. For example, without such integration, the system 100 may update a research site's subject (e.g., patient) enrollment metrics in a manual fashion—i.e., researchers manually obtain this enrollment data from a study record housed outside the system 100. However, with this integration function enabled, the system 100 can link (e.g., via open-sourced application programming interface (API) communication) to external CTMS systems. In one embodiment, the system 100 then uses these links to route data from the CTMS system to the system 100 to automatically update data (e.g., enrollment data) in the system 100 seamlessly without traditional manual processes.

Further, various elements of the system 100 may communicate with each other through a communication network 103. The communication network 103 of system 100 includes one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular communication network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (Wi-Fi), wireless LAN (WLAN), Bluetooth®, Internet Protocol (IP) data casting, satellite, mobile ad-hoc network (MANET), vehicle controller area network (CAN bus), and the like, or any combination thereof.

In one embodiment, the clinical trial management platform 105 may be a platform with multiple interconnected components. The clinical trial management platform 105 may include one or more servers, intelligent networking devices, computing devices, components and corresponding software for managing various stages of clinical trials and/or research studies. In one embodiment, the clinical trial management platform 105 may manage pipelines of clinical research sites on clinical trials and/or research studies. In another embodiment, the clinical trial management platform 105 may automatically date and time stamp key performance benchmarks or metrics, thereby enabling research sites to track and gain insight into their site's operating performance. In a further embodiment, the clinical trial management platform 105 may permit reporting of site performance metrics and enables clinical research sites to tout their capabilities at managing clinical trials to their drug company sponsor clients and CRO's.

In one embodiment, the clinical trial management platform 105 may contain multiple proprietary workflow that are governed by rules to fully automate the enormously complex process of tracking a potential research study from initial lead to final award letter and study start-up. In one example embodiment, the workflow may be a flowchart of the process for carrying out the tasks for automation of the complex process of tracking clinical trials and research work, and improve efficiency of their operations. The workflow may comprise of a set of methods to manage and control execution of specified task, and determine when a process is ready to move to a next step in the pipeline. By way of example, one or more users (e.g., researchers, managers, clinical research sites, etc.) may use any communications enabled computing device to access the clinical trial management platform 105 and/or the clinical research site database 101.

In one embodiment, users access the functions of the clinical trial management platform 105 through user devices 113 and/or devices 115. For example, the devices 113 and 115 may execute respective applications or client software (e.g., native applications, web-based applications, browser front-end applications, etc.) to interact with the clinical trial management platform 105 and/or the clinical research site databases 101. In one embodiment, the devices 113 and 115 are any type of mobile terminal, fixed terminal, or portable terminal including a mobile handset, station, unit, device, multimedia computer, multimedia tablet, Internet node, communicator, desktop computer, laptop computer, notebook computer, netbook computer, tablet computer, personal communication system (PCS) device, personal digital assistants (PDAs), audio/video player, digital camera/camcorder, positioning device, television receiver, radio broadcast receiver, electronic book device, game device, or any combination thereof, including the accessories and peripherals of these devices, or any combination thereof. It is also contemplated that the devices 113 and 115 can support any type of interface to the user (such as “wearable” circuitry, etc.).

The services platform 109 may include any type of service. By way of example, the services platform 109 may include content provisioning services/application, application services/application, storage services/application, contextual information determination services/application, management service/application, etc. In one embodiment, the services platform 109 may interact with the clinical trial management platform 105 and the content provider 111 to supplement or aid in the processing of the content information.

The content providers 111 may provide content to the clinical trial management platform 105. The content provided may be any type of content, such as, image content, textual content, audio content, video content, etc. In one embodiment, the content provider 111 may provide or supplement the content (e.g., audio, video, images, etc.) provisioning services/application, application services/application, storage services/application, contextual information determination services/application. In one embodiment, the content provider 111 may also store content associated with the clinical trial management platform 105, and the services platform 109. In another embodiment, the content provider 111 may manage access to a central repository of data, and offer a consistent, standard interface to data, such as, a repository of clinical trials or research studies for one or more clinical research sites. Any known or still developing methods, techniques or processes for assigning at least one location to at least one contact may be employed by the clinical trial management platform 105.

By way of example, the clinical trial management platform 105 communicate with database 101 and other components of the communication network 107 using well known, new or still developing protocols. In this context, a protocol includes a set of rules defining how the network nodes within the communication network 103 interact with each other based on information sent over the communication links. The protocols are effective at different layers of operation within each node, from generating and receiving physical signals of various types, to selecting a link for transferring those signals, to the format of information indicated by those signals, to identifying which software application executing on a computer system sends or receives the information. The conceptually different layers of protocols for exchanging information over a network are described in the Open Systems Interconnection (OSI) Reference Model.

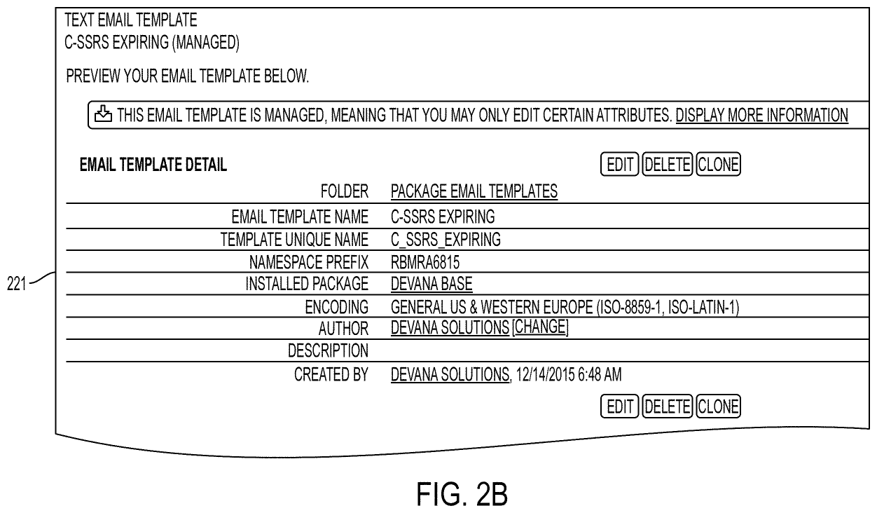

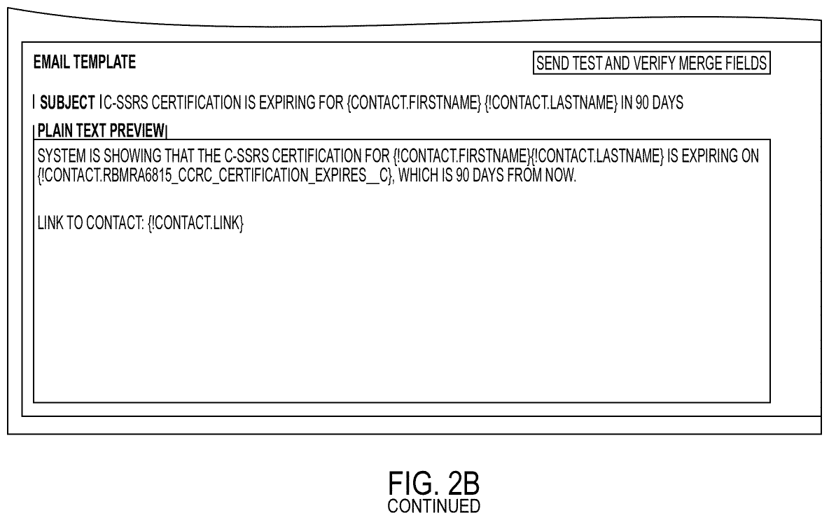

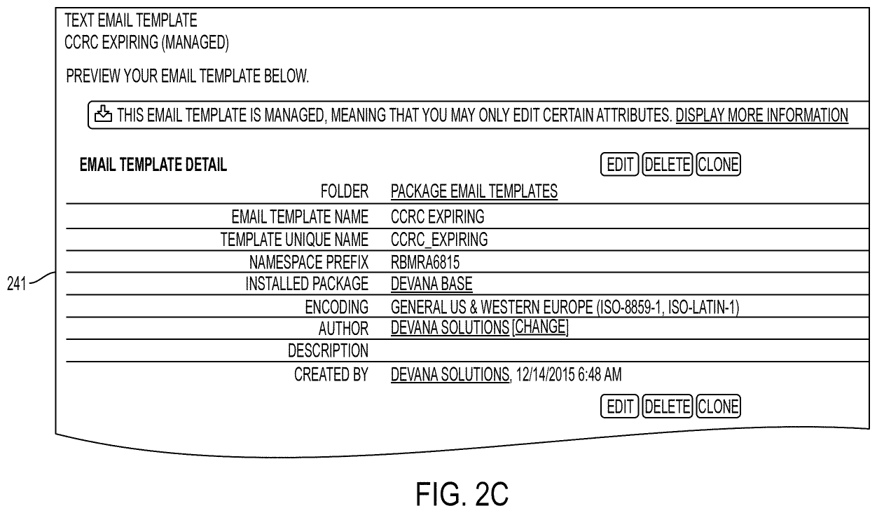

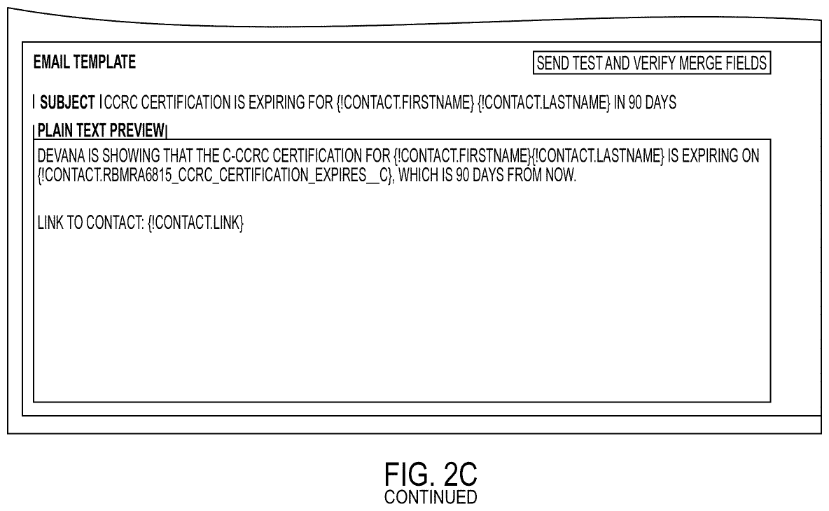

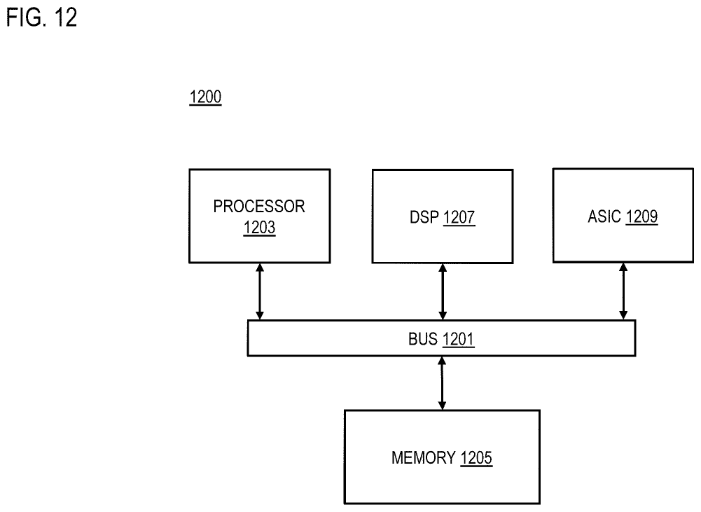

FIG. 2A is a flowchart of a process for generating a user interface for capturing time and performance metrics of a clinical trial process based on workflow rules, according to one embodiment. In one embodiment, the clinical trial management platform 105 performs the process 200 and is implemented in, for instance, a chip set including a processor and a memory as shown in FIG. 12. The example of FIG. 2 assumes that a clinical trial data record has already been created by the clinical trial management platform 105.

In step 201, the clinical trial management platform 105 receives an input via a user interface element of a user interface of the clinical trial management platform 105. For example, the input can specify an edit or an update to a data record associated with tracking a clinical trial process. In one embodiment, the edit or update initiates a dynamic update process which consists, for instance of three types of updates. For example, the first type of update determines the stages available for a particular record type of the data record, the second type determines what user interface elements (e.g., buttons, options, etc.) are available on the edit screen or edit mode of the user interface, and the third type determines what fields are available on the edit screen of the user interface. The types of updates are discussed in more detail below.

In one embodiment, the data record includes a plurality of fields, and wherein each of the plurality of fields represents a respective stage of the clinical trial process. Table 1 below lists examples of stages and the fields of the trial data record that represent the stages (e.g., including pre-award stages, award stages, study stages, and study completion stages). Table 1 is provided by way of illustration and not limitation.

TABLE 1
Stage
ClinicalTrials.gov (e.g., published availability of a clinical trial)
Long Shot
Pre-Award
Pipeline
Confidential Disclosure Agreement (CDA) Received
CDA Signed
Feasibility Received
Feasibility Completed
Pre-Study Visit (PSV) Requested
PSV Date Set
PSV Completed
Not Interested in Trial
Trial Not Received
Start Up Stage
Awarded but Passed
Executed/Enrollment Stage
Currently Running
Trial Completed

In step 203, the clinical trial management platform 105 determines a workflow rule associated with the user interface element, wherein the workflow rule specifies one or more criteria for initiating a capture of a timing metric, a performance metric, or a combination thereof associated with the clinical trial process. In one embodiment, the clinical trial management platform 105 can assign workflow rules to different actions that a user can take with respect to user interface elements and/or the user interface of the clinical trial management platform 105.

In step 205, the clinical trial management platform 105 initiates the capture of the timing metric, the performance metric, or a combination thereof based on an evaluation of the input against the one or more criteria. For example, an action such as “checking a Trial Milestone checkbox” (an example of a user interface element) in the user interface can trigger an evaluation of a workflow rule associated with the checkbox or data record field associated with the checkbox. In one embodiment, if the evaluation of the rule returns a true value or otherwise meets a specified criterion, an action occurs that automatically records timing or performance metrics. Table 2 below provides examples of workflow rules, criteria for triggering an associated action, and the action themselves. In this example, the workflow rules are named similarly to their associated data fields. As described above, each field represents, for instance, a stage in the clinical trial process. It is noted that Table 2 is provided by way of illustration and not limitation to the embodiments described herein with respect to workflow rules.

TABLE 2

| Workflow Rule | Criteria and Action |
|---|---|
| Trial-Budget Agreed Date | Evaluate the rule when a record is created, and any time it’s |
| | edited to subsequently meet criteria. |
| | Rule Criteria: Trial: Budget Agreed ToEQUALSTrue |
| | Action: Trial-Budget Agreed Date field is updated to TODAY ( ) |
| Trial-Budget Received Date | Evaluate the rule when a record is created, and any time it’s |
| | edited to subsequently meet criteria |
| | Rule Criteria: Trial: Budget ReceivedEQUALSTrue |
| | Action: Trial-Budget Received Date field is updated to TODAY ( ) |
| Trial-Budget Rev 1 Sub | Evaluate the rule when a record is created, and any time it's |
| (Managed) | edited to subsequently meet criteria |
| | Rule Criteria: Trial: Budget Revision 1 SubmittedEQUALSTrue |
| | Action: Trial-Budget Rev 1 Sub field is updated to TODAY ( ) |
| Trial-Contract Review | Evaluate the rule when a record is created, and any time it's |
| Complete | edited to subsequently meet criteria |
| | Rule Criteria: Trial: Contract Review CompleteEQUALSTrue |
| | Action: Update Trial-Contract Review Complete field to |
| | TODAY( ) |
| Trial-First Seen | Evaluate the rule when a record is created, and any time it's |
| | edited to subsequently meet criteria |
| | Rule Criteria: Trial: Created DateNOT EQUAL TOnull |
| | Action: Update Trial-First Seen field to TODAY( ) |
| Trial-CDA Partially Executed | Evaluate the rule when a record is created, and any time it's |
| | edited to subsequently meet criteria |
| | Rule Criteria: Trial: CDA Partially EecutedEQUALSTrue |
| | Action: Update field Trial-CDA Partially Executed to |
| | TODAY( ) |
| Trial-Budget Resp 1 Rec | Evaluate the rule when a record is created, and any time it's |
| | edited to subsequently meet criteria |
| | Rule Criteria: Trial: Budget Response 1 ReceivedEQUALSTrue |
| | Action: Update Trial-Budget Resp 1 Rec field to TODAY( ) |
| Trial-Budget Resp 2 Rec | Evaluate the rule when a record is created, and any time it's |
| | edited to subsequently meet criteria |
| | Rule Criteria: Trial: Budget Response 2 ReceivedEQUALSTrue |
| | Action: Update Trial-Budget Resp 2 Rec field to TODAY( ) |
| Trial-Budget Resp 3 Rec | Evaluate the rule when a record is created, and any time it's |
| | edited to subsequently meet criteria |
| | Rule Criteria: Trial: Budget Response 3 ReceivedEQUALSTrue |
| | Action: Update Trial-Budget Resp 3 Rec field to TODAY( ) |
| Trial-Budget Rev 2 Sub | Evaluate the rule when a record is created, and any time it's |
| | edited to subsequently meet criteria |
| | Rule Criteria: Trial: Budget Revision 2 SubmittedEQUALSTrue |
| | Action: Update Trial-Budget Rev 2 Sub field to TODAY( ) |
| Trial-Budget Rev 3 Sub | Evaluate the rule when a record is created, and any time it's |
| | edited to subsequently meet criteria |
| | Rule Criteria: Trial: Budget Revision 3 SubmittedEQUALSTrue |
| | Action: Update Trial-Budget Rev 3 Sub field to TODAY( ) |
| Trial-CDA Received | Evaluate the rule when a record is created, and any time it's |
| | edited to subsequently meet criteria |
| | Rule Criteria: |
| | (Trial: CDA ReceivedEQUALSTrue) AND (Trial: CDA Fully |
| | ExecutedEQUALSFalse) |
| | Action: Update Trial-CDA Received Date field to TODAY( ) |
| | Action: Update Trial-CDA Received Stage field to CDA |
| | Received |
| Trial-CDA Signed | Evaluate the rule when a record is created, and any time it's |
| | edited to subsequently meet criteria |
| | Rule Criteria: Trial: CDA Fully EecutedEQUALSTrue |
| | Action: Update Trial-CDA Signed Date field to TODAY( ) |
| | Action: Update Trial-CDA Signed Stage field to CDA Signed |
| Trial-Contract Agreed To | Evaluate the rule when a record is created, and any time it's |
| | edited to subsequently meet criteria |
| | Rule Criteria: Trial: Contract Agreed ToEQUALSTrue |
| | Action: Update Trial-Contract Agreed To field to TODAY( ) |
| Trial-Contract Executed | Evaluate the rule when a record is created, and any time it's |
| | edited to subsequently meet criteria |
| | Rule Criteria: Trial: Contract Executed by SiteEQUALSTrue |
| | Action: Update Trial-Contract Executed field to TODAY( ) |
| Trial-Contract Received | Evaluate the rule when a record is created, and any time it's |
| | edited to subsequently meet criteria |
| | Rule Criteria: Trial: Contract ReceivedEQUALSTrue |
| | Action: Update Trial-Contract Received field to TODAY( ) |
| Trial-Start Date | Evaluate the rule when a record is created, and any time it's |
| | edited to subsequently meet criteria |
| | Rule Criteria: Trial: START TRIALNOT EQUAL TOnul |
| | Action: Update Trial-Start Date field to TODAY( ) |
| Trial-Contract Returned | Evaluate the rule when a record is created, and any time it's |
| | edited to subsequently meet criteria |
| | Rule Criteria: Trial: Contract ReturnedEQUALSTrue |
| | Action: Update Trial-Contract Returned field to TODAY( ) |
| Trial-Contract Sent Review | Evaluate the rule when a record is created, and any time it's |
| | edited to subsequently meet criteria |
| | Rule Criteria: Trial: Contract Sent for ReviewEQUALSTrue |
| | Action: Update Trial-Contract Sent Review field to TODAY( ) |
| Trial-Executed Contract | Evaluate the rule when a record is created, and any time it's |
| Received | edited to subsequently meet criteria |
| | Rule Criteria: Trial: Fully Executed Contract in |
| | HandEQUALSTrue |
| | Action: Update Trial-Currently Running Date field to |
| | TODAY( ) |
| | Action: Update Trial-Currently Running Stage field to |
| | Executed/Enrollment Stage |
| Trial-Feasibility Completed | Evaluate the rule when a record is created, and any time it's |
| | edited to subsequently meet criteria |
| | Rule Criteria: Trial: Feasibility CompletedEQUALSTrue |
| | Action: Update Trial-Feasibility Completed Date field to |
| | TODAY( ) |
| | Action: Update Trial-Feasibility Completed Stage field to |
| | Feasibility Completed |
| Trial-Feasibility Received | Evaluate the rule when a record is created, and any time it's |
| | edited to subsequently meet criteria |
| | Rule Criteria: (Trial: Feasibility ReceivedEQUALSTrue) AND |
| | (Trial: Feasibility CompletedEQUALSFalse) |
| | Action: Update Trial-Feasibility Received Date field to |
| | TODAY( ) |
| | Action: Update Trial-Feasibility Received Stage field to |
| | Feasibility Received |
| Trial-Intramural Research | Evaluate the rule when a record is created, and any time it's |
| Program (IRP) Confirmed | edited to subsequently meet criteria |
| | Rule Criteria: Trial: Initial Regulatory Confirmed |
| | CompleteEQUALSTrue |
| | Action: Update Trial-IRP Confirmed field to TODAY( ) |
| Trial-IRP Started | Evaluate the rule when a record is created, and any time it's |
| | edited to subsequently meet criteria |
| | Rule Criteria: Trial: Initial Regulatory StartedEQUALSTrue |
| | Action: Update field Trial-IRP Started to TODAY( ) |
| Trial-IRP Submitted | Evaluate the rule when a record is created, and any time it's |
| | edited to subsequently meet criteria |
| | Rule Criteria: Trial: Initial Regulatory SubmittedEQUALSTrue |
| | Action: Update field to TODAY( ) |
| Trial-PSV Completed | Evaluate the rule when a record is created, and any time it's |
| | edited to subsequently meet criteria |
| | Rule Criteria: Trial: PSV CompleteEQUALSTrue |
| | Action: Update Trial-PSV Completed Date field to TODAY( ) |
| | Action: Update Trial-PSV Completed Stage field to PSV |
| | Completed |
| Trial-PSV Confirmed | Evaluate the rule when a record is created, and any time it's |
| | edited to subsequently meet criteria |
| | Rule Criteria: (Trial: PSV Date ConfirmedEQUALSTrue) AND |
| | (Trial: PSV CompleteEQUALSFalse) |
| | Action: Update Trial-PSV Confirmed Date field to TODAY( ) |
| | Action: Update Trial-PSV Confirmed Stage field to PSV Date |
| | Set |
| Trial-PSV Requested | Evaluate the rule when a record is created, and any time it's |
| | edited to subsequently meet criteria |
| | Rule Criteria: (Trial: PSV RequestedEQUALSTrue) AND |
| | (Trial: PSV Date ConfirmedEQUALSFalse) AND (Trial: PSV |
| | CompleteEQUALSFalse) |
| | Action: Update Trial-PSV Requested Date field to TODAY( ) |
| | Action: Update Trial-PSV Requested Stage field to PSV |
| | Requested |
| Trial-Import Created Date | Evaluate the rule when a record is created, and any time it's |
| | edited to subsequently meet criteria |
| | Rule Criteria: Trial: Created DateNOT EQUAL TOnull |
| | Action: Update Trial-Import Created Date field to |
| | DATEVALUE (CreatedDate) |

As shown above, in many of the examples of Table 1, the action associated with workflow rules results in automatically capturing a timing metric associated with a stage of the clinical trial. In one embodiment, the timing metric represents a completion date of the respective stage of the clinical trial process. For example, the timing metric can be recorded as the date corresponding to when the user interacts with a user interface element (e.g., selecting a checkbox). In addition or alternatively, the clinical trial management platform 105 can present another prompt in the user interface that requests a manual user entry of the timing metric (e.g., a completion date of the stage if the completion of the stage occurred on a date other than the date of interaction with the user interface element). In yet another embodiment, clinical trial management platform 105 can query another system or database for the completion date to determine the timing metric.

In one embodiment, the clinical trial management platform 105 calculates a time-based performance metric based on a time period between the completion date of a respective stage of the clinical trial process and a prior completion date of a prior respective stage of the clinical trial process. By way of example, the time-based performance metric can be represented simply as the number of days in the calculated time period. In other embodiments, the clinical trial management platform 105 can compare or normalize the time-based performance metric against historic performance metrics for the same or similar stages of the clinical trial process. In addition, the performance metric can be compared or normalized to performance metrics collected only from the same research site, other research sites, a population of research sites, and the like.

In one embodiment, the clinical trial management platform 105 can determine performance metrics based on criteria other than time. For example, the platform 105 can determine a performance metric based on a cost metric for completing the respective stage of the clinical trial process. For example, the platform 105 can query or otherwise interact with a budgeting tool of the system 100 to determine budgeting and/or cost information associated particular stages of the clinical trial process. The cost information can include costs-per-procedure to perform as part of a study, as well as overhead costs such as marketing or proposal preparation costs. In one embodiment, the performance metric is then further based on the cost metric. As with the time-based performance metric, the cost-based performance metric can also be compared or normalized against historical metrics, standard industry metrics, etc.

It is contemplated that time and costs are provided only as examples and not limitations of a performance metric. It is contemplated that any other performance metric (e.g., manpower level, recorded evaluations or ratings of research site performance, resulting regulatory approval, etc.) can be used according to the various embodiments described herein.

In step 207, the clinical trial management platform 105 stores the timing metric, the performance metric, or a combination thereof to update the data record.

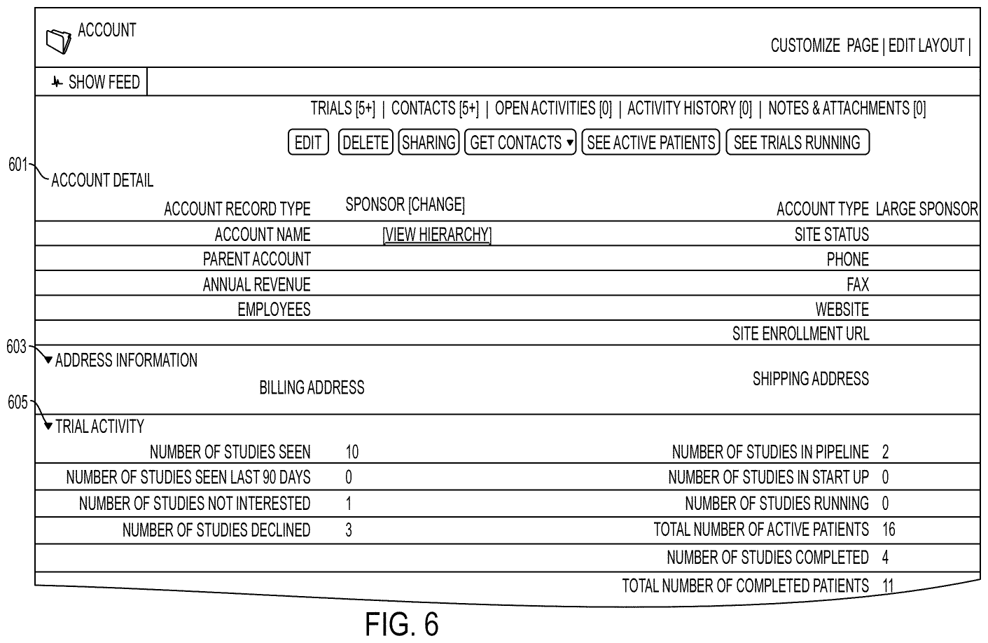

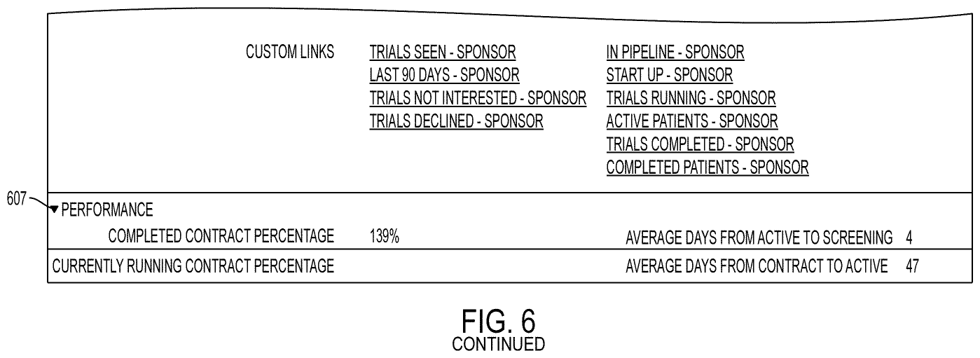

In step 209, the clinical trial management platform 105 updates a plurality of user interface elements that are visible in the user interface based on the updated data record. For example, in one embodiment, the clinical trial management platform 105 determines a record type of the data record based on a state of the data record. In one embodiment, the state represents a relationship between the clinical trial process and a site that is a participant or a potential participant of the clinical trial process. Examples of the states of the data record or clinical trial are discussed with respect to FIG. 6 below and include: (1) Prior to Site Being Selected; (2) Site Selected/Award Letter Received; (3) Trial Received—Running; (4) Trial Completed; (5) Trial Awarded but Passed; (6) Historical Trial; and (7) Site Not Interested in Trial. The plurality of user interface elements that are visible in the user interface is further based on the record type of the data record. In other words, when a user selects a record type for a trial data record, the clinical trial management platform 105 dynamically displays what stages or fields of the data record are available for the record type reflecting the state of the data record. Table 3 below illustrates a matrix showing the example relationships between the record type and the available stages. The illustrated relationships are provided as examples and are not intended as limitations. As shown in Table 3, the listed record types correspond to the seven states listed above in this paragraph.

TABLE 3

| Stage | Type 1 | Type 2 | Type 3 | Type 4 | Type 5 | Type 6 | Type 7 |
|---|---|---|---|---|---|---|---|
| ClinicalTrials.gov | Visible | Hidden | Hidden | Hidden | Hidden | Hidden | Hidden |
| Long Shot | Visible | Hidden | Hidden | Hidden | Hidden | Hidden | Hidden |
| Pre-Award | Visible | Hidden | Hidden | Hidden | Hidden | Hidden | Hidden |
| Pipeline | Visible | Hidden | Hidden | Hidden | Hidden | Hidden | Hidden |
| CDA Received | Visible | Hidden | Hidden | Hidden | Hidden | Hidden | Hidden |
| CDA Signed | Visible | Hidden | Hidden | Hidden | Hidden | Hidden | Hidden |
| Feasibility Received | Visible | Hidden | Hidden | Hidden | Hidden | Hidden | Hidden |
| Feasibility Completed | Visible | Hidden | Hidden | Hidden | Hidden | Hidden | Hidden |
| PSV Requested | Visible | Hidden | Hidden | Hidden | Hidden | Hidden | Hidden |
| PSV Date Set | Visible | Hidden | Hidden | Hidden | Hidden | Hidden | Hidden |
| PSV Completed | Visible | Hidden | Hidden | Hidden | Hidden | Hidden | Hidden |
| Not Interested in Trial | Visible | Hidden | Hidden | Hidden | Hidden | Hidden | Visible |
| Trial Not Received | Visible | Hidden | Hidden | Hidden | Hidden | Hidden | Visible |
| Start Up Stage | Visible | Visible | Hidden | Hidden | Hidden | Hidden | Hidden |
| Awarded but Passed | Visible | Visible | Hidden | Hidden | Visible | Hidden | Hidden |
| Executed/Enrollment | Visible | Visible | Hidden | Hidden | Hidden | Hidden | Hidden |
| Stage | | | | | | | |
| Currently Running | Visible | Hidden | Visible | Hidden | Hidden | Hidden | Hidden |
| Trial Completed | Visible | Hidden | Hidden | Visible | Hidden | Visible | Hidden |

In one embodiment, the clinical trial management platform 105 determines a plurality of editing user interface elements that are visible in an editing mode of the user interface based on the record type. In addition to dynamically changing the visible or available stages or fields based on a record type of the trial data record, the record type can also determine what buttons or other user interface elements are visible or available in an editing mode or screen of the platform user interface. Table 4 below illustrates a matrix outlining what editing user interface elements or buttons are visible or available for each of the seven record types described above. Table 4 is provided as an example and is not intended as a limitation.

TABLE 4

| Button/UI Element | Type 1 | Type 2 | Type 3 | Type 4 | Type 5 | Type 6 | Type 7 |
|---|---|---|---|---|---|---|---|
| Edit | Visible | Visible | Visible | Visible | Visible | Visible | Visible |
| Delete | Visible | Visible | Visible | Visible | Visible | Visible | Visible |
| Clone | Visible | Visible | Visible | Visible | Visible | Visible | Visible |
| Awarded Trial | Visible | Hidden | Hidden | Hidden | Visible | Hidden | Hidden |
| Not Awarded Trial | Visible | Hidden | Hidden | Hidden | Hidden | Hidden | Hidden |
| Discard Trial | Visible | Hidden | Hidden | Hidden | Hidden | Hidden | Hidden |
| Indication | Visible | Visible | Visible | Visible | Visible | Visible | Visible |
| Performance | | | | | | | |
| All Trials in Same | Visible | Visible | Visible | Visible | Visible | Visible | Visible |
| Indication | | | | | | | |
| Trial Complete | Hidden | Hidden | Visible | Hidden | Hidden | Hidden | Hidden |
| Pass on Trial | Hidden | Visible | Visible | Hidden | Hidden | Hidden | Hidden |
| Reactivate Trial | Hidden | Visible | Hidden | Hidden | Visible | Hidden | Visible |
| Start Trial | Hidden | Visible | Hidden | Hidden | Hidden | Hidden | Hidden |

In one embodiment, once a trial data record is created and the appropriate record type has been selected, the user can now edit the trial based on the buttons or user interface elements that are visible according to Table 4. For example, there are many dynamic updates that can occur while having the trial user interface in edit mode. In one embodiment, the user interface screen can be split into sections and each section, along with their corresponding user interface elements (e.g., checkboxes, lists, text fields, etc.) can be dynamically displayed based upon the selected or determined record type. By way of example and not limitation, the sections of the editing mode of the trial user interface may include one or more of the following:
Trial Detail;
Institutional Review Board (IRB);
Updates;
Recruitment Numbers;
Dashboard;
Contract Timelines;
Initial Regulatory Timelines;
Budget Negotiation;
Trial Milestones;
Feasibility Reminder;
Trial Timelines;
Site Timelines;
Sponsor/CRO Timelines;
Follow Up Timelines;
User Detail;
Trial Contacts;
Regulatory/Site Start Up(s);
Open Activities;
Activity History;
Site Activity and Enrollment;
Electronic Data Capture (EDC)/Interactive Voice Response System (IVRS)/Interactive Web Response System (IWRS);
Close Outs;
Advertising Budgets;
Notes & Attachments; and
Trial History.

In one embodiment, as noted above, each section of the editing user interface contains a combination of user interface elements (e.g., checkboxes, lists, text fields, etc.). The sections and/or fields are dynamically visible or hidden based on the record type selected or determined for the trial data record.

In one embodiment, as the user interacts with the various visible user interface elements presented in each section of the trial user interface, the stage and/or state of the trial is dynamically updated as well as other field values depending on the stage or field selected. For example, when a user selects the CDA Received checkbox the stage field is updated to “CDA Received” and the Calculated Close date is automatically calculated to a future date in time. Table 5 below illustrates, by way of example and not limitation, dynamic updates that occur for Record Type 1 (“Prior to Site Being Selected”). In one embodiment, even if not shown in Table 5, the time and/or performance metric can be recorded by the clinical trial management platform 105 based on a user interaction at each listed stage or field.

TABLE 5

| Stage/Field | Type 1-Prior to Site Being Selected |
|---|---|
| CDA | Received When user selects this checkbox: |
| | 1. Stage field is updated to “CDA Received” |
| | 2. Calculated Close Date is auto calculated |
| CDA Fully | When user selects this checkbox: |
| Executed | 1. Stage field is updated to “CDA Signed” |
| Feasibility | When user selects this checkbox: |
| Received | 1. Stage field is updated to “Feasibility Received” |
| Feasibility | When user selects this checkbox: |
| Completed | 1. Stage field is updated to “Feasibility Completed” |
| PSV Requested | When user selects this checkbox: |
| | 1. Stage field is updated to “PSV Requested” |
| PSV Date | When user selects this checkbox: |
| Confirmed | 1. Stage field is updated to “PSV Date Set” |
| PSV Completed | When user selects this checkbox: |
| | 1. Stage field is updated to “PSV Completed” |
| | 2. Calculated Close Date is auto calculated |
| Award Letter | When user selects this checkbox: |
| Received | 1. Record type converts to Site Selected/Award |
| | Letter Received |
| | 2. Calculated Close Date is auto calculated |
| | 3. Actual Close Date field populates |

Table 6 below illustrates, by way of example and not limitation, dynamic updates that occur for Record Type 2 (“Site Selected/Award Letter Received”). In one embodiment, even if not shown in Table 6, the time and/or performance metric can be recorded by the clinical trial management platform 105 based on a user interaction at each listed stage or field.

TABLE 6

| Stage/Field | Type 3-Site Selected/Award Letter Received |
|---|---|
| Contract Received | When user selects this checkbox: |
| | 1. Timing and/or performance metric recorded |
| Contract Sent for Review | When user selects this checkbox: |
| | 1. Timing and/or performance metric recorded |
| Contract Review Completed | When user selects this checkbox: |
| | 1. Timing and/or performance metric recorded |
| Contract Returned | When user selects this checkbox: |
| | 1. Timing and/or performance metric recorded |
| Contract Agreed To | When user selects this checkbox: |
| | 1. Timing and/or performance metric recorded |
| Contract Executed by Site | When user selects this checkbox: |
| | 1. Timing and/or performance metric recorded |
| Fully Executed Contract | When user selects this checkbox: |
| in Hand | 1. Stage field is updated to “Executed/Enrollment Stage” |
| Budget Received | When user selects this checkbox: |
| | 1. Budget Status field is updated to “Budget in House” |
| Budget Revision 1 | When user selects this checkbox: |
| Submitted | 1. Budget Status field is updated to “In Negotiation” |
| Budget Response 1 | When user selects this checkbox: |
| Received | 1. Timing and/or performance metric recorded |
| Budget Revision 2 | When user selects this checkbox: |
| Submitted | 1. Timing and/or performance metric recorded |
| Budget Response 2 | When user selects this checkbox: |
| Received | 1. Timing and/or performance metric recorded |
| Budget Revision 3 | When user selects this checkbox: |
| Submitted | 1. Timing and/or performance metric recorded |
| Budget Response 3 | When user selects this checkbox: |
| Received | 1. Timing and/or performance metric recorded |
| Budget Agreed To | When user selects this checkbox: |
| | 1. Budget Status field is updated to “Budget Agreed To” |
| Initial Regulatory Received | When user selects this checkbox: |
| | 1. Initial Regulatory Status field is updated to |
| | “Initial Regulatory in House” |
| Initial Regulatory Started | When user selects this checkbox: |
| | 1. Initial Regulatory Status field is updated to |
| | “Initial Regulatory Started” |
| Initial Regulatory Submitted | When user selects this checkbox: |
| | 1. Initial Regulatory Status field is updated to |
| | “Initial Regulatory Submitted” |
| Initial Regulatory Confirmed | When user selects this checkbox: |
| Complete | 1. Initial Regulatory Status field is updated to |
| | “Initial Regulatory Complete” |
| Start Trial | When user selects this checkbox or the action button |
| | of the same name in the user interface: |
| | 1. Stage changes to “Currently Running” |
| | 2. Enrollment Status changes to “Enrollment Pending” |
| | 3. Trial Start Date field enters date |

In step 211, the clinical trial management platform 105 optionally sets reminder notifications for a subsequent stage of the clinical trial process and/or a future action related to the clinical trial process. In one embodiment, the clinical trial management platform 105 determines a calculated future date for completing a subsequent stage of the clinical trial process. In one embodiment, the clinical trial management platform 105 determines the calculated future date based on historical timing metrics, historical performance metrics, or a combination thereof associated with the clinical trial process, one or more similar clinical trial processes, or a combination thereof.

The platform 105 then automatically sets a reminder notification of the calculated future date, and presents the reminder notification associated with the subsequent stage of the clinical trial process in the platform's user interface. Table 7 below illustrates example workflow rules for automatically creating email alerts or notifications according to the embodiments described herein. More specifically, Table 7 illustrates workflow rules for notifying a contact about upcoming expirations of, for instance, certain training or certifications (e.g., Columbia Suicide Severity Rating Scale (C-SSRS) screener training, Certified Clinical Researcher Coordinator (CCRC) certification, etc.).

TABLE 7

| Workflow | |
|---|---|
| Rule | Criteria and Action |
| C-SSRS | Email Notification is sent when the CCRC certification |
| Expiring | for a contact is expiring to alert the Account Owner |
| Email | Action: Create email using C-SSRS Expiring Email |
| | Template (see example template of FIG. 2B) |
| CCRC | Email Notification is sent when the C-SSRS training |
| Expiring | certification for a contact is expiring to alert the |
| Email | Account Owner |
| | Action: Create email using C-SSRS Expiring Email |
| | Template (see example template of FIG. 2C) |

FIGS. 2B and 2C depict example user interfaces for presenting an email template to send the notification alerts about future expirations of the C-SSRS and CCRC respectively. As shown in FIG. 2B, a user interface 221 presents user interface elements that identify the template as a C-SSRS Expiring template and presents the parameters and notification text for generating a reminder. In this example, the notification is configured to be sent 90 days before the expiration date of the C-SSRS certification. The parameters of the template include a contact (e.g., a link to an entry in a contact database such as in a CRM system) for whom the C-SSRS certification is expiring and the expiration date. Similarly, FIG. 2C presents a user interface 241 depicting user interface elements that identify the template as a CCRC Expiring template and presents associated parameters and notification text for generating or setting a reminder. In the example of FIG. 2C, the notification data is also 90 days before the expiration date of the CCRC. It is noted that 90 days is provided only as an example, and the system 100 can be configured to generate or set reminders based on any criteria or date.

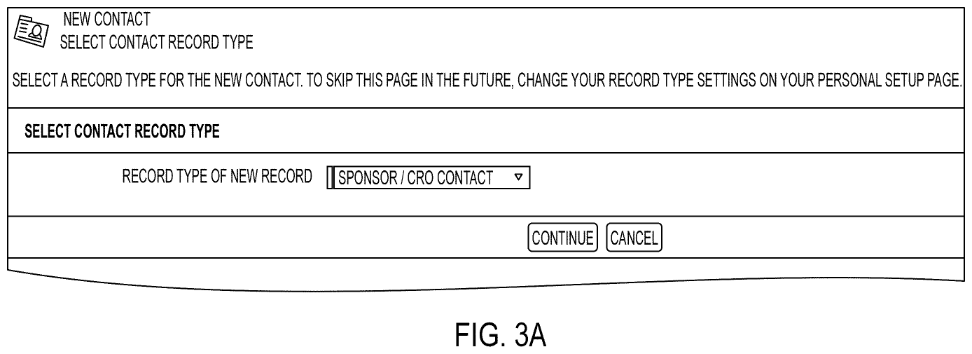

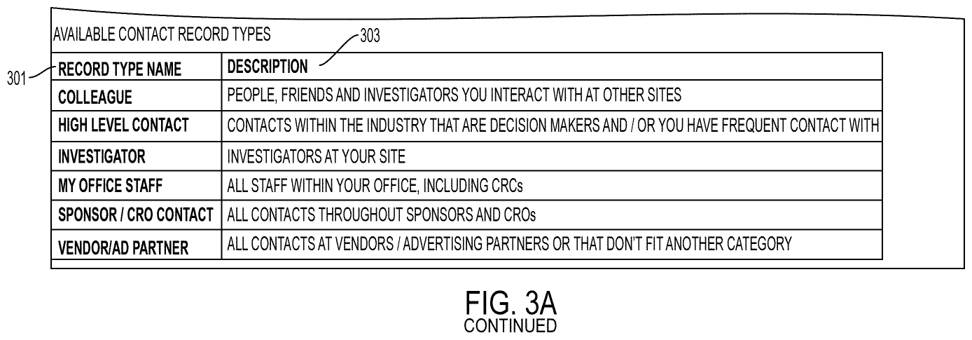

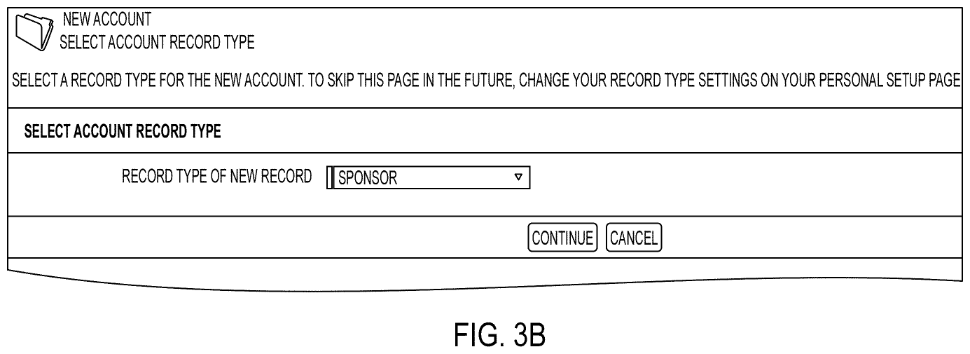

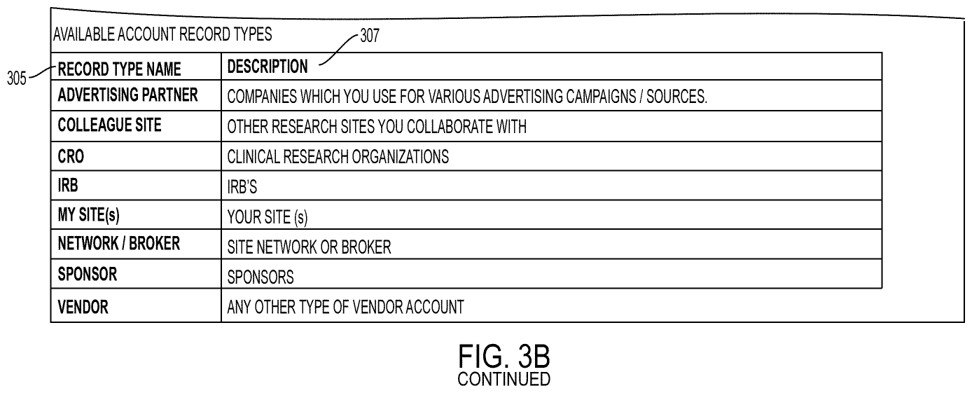

FIG. 3A is a diagram that represents available contact record types, according to one example embodiment. In one embodiment, the contact record types include information on the record type name 301 with their description 303. In one example embodiment, the record type 301 “Colleague” may have a description 303 of “people, friends and investigators one interacts with at one or more research sites.” In another example embodiment, the record type 301 “High Level Contact” may have a description 303 of “Contacts within the industry that are decision makers and/or you have frequent contact with.”

FIG. 3B is a diagram that represents available account record types, according to one example embodiment. In one embodiment, the account record type includes information on the record type name 305 with their description 307. In one example embodiment, the record type 305 “Advertising Partner” may have a description 307 of “companies which you use for various advertising campaigns/sources.” In another example embodiment, the record type 305 “Colleague Site” may have a description 207 of “other research site one collaborates with.”

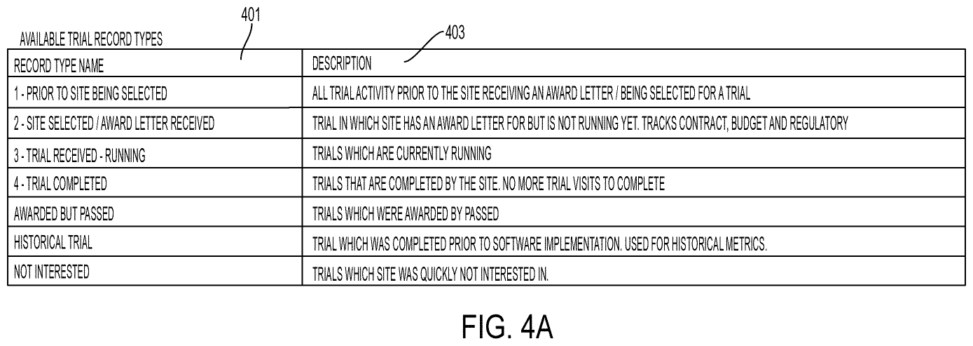

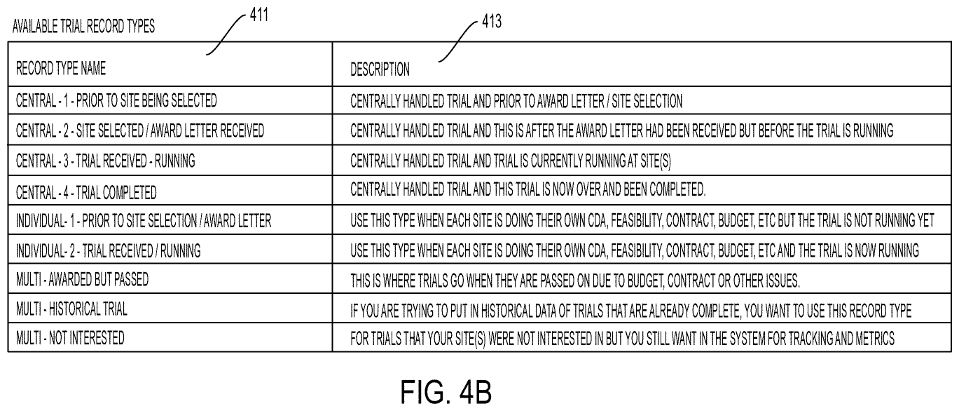

FIGS. 4A and 4B are diagrams that represent the available trial record types respectively for a single site use case and a multiple site use case, according to various embodiments. In other words, the system 100 can be used to manage the clinical trial process for an entity with a single participating research site, as well as for an entity with multiple participating research sites. Accordingly, in one embodiment, the system 100 can apply different trial record types depending on the use case (e.g., single site or multiple site).

FIG. 4A presents, by way of illustration and not limitation, the available trial record types 401 and their descriptions 403 for a use case where there is only a single research site. In one embodiment, under a single site use case, all of the record types apply to the single site. As shown, each record type 401 is associated with a description 403. Examples of record types 401 and descriptions 403 include: (a) “1—Prior to Site Being Selected” which is a record type associated with all trial activity prior to the site receiving an award letter or being selected for a trial; (b) “2—Site Selected/Award Letter Received” which is a record type associated with a trial in which a site has an award letter but the trial is not yet running, and includes pre-running activities such as contract, budget, and regulatory related activities or milestones; (c) “3—Trial Received—Running” which is a record type associated with trials that are currently running at the single site; (d) “4—Trial Completed” which is a record type associated with trials that are completed by the single site, and for which there are no more trial visits to complete; (e) “Awarded but Passed” which is a record type associated with trials which were awarded to the single site but passed by the site; (f) “Historical Trial” which is a record type associated with trials completed by the site prior to being tracked by the system 100 and/or otherwise used for calculating historical metrics; and (g) “Not Interested” which is a record type associated with trials in which the single site was not interested in participating.

FIG. 4B presents, by way of illustration and not limitation, the available trial record types 411 and their descriptions 413 for a use case where an entity is managing the trial process for multiple participating sites (e.g., multiple sites of one entity that are soliciting participation in a trial as a group). As shown, the record types 411 for this use case can be categorized according to whether a trial and/or its associated activities or milestones are centrally handled (e.g., handled by a central entity on behalf of the sites), individually applicable to each participating site of the multiple sites, or applicable to the multiple sites as a whole. For example, centrally handled record types include: (a) “Central—1—Prior to Site Being Selected” which is a record type associated trial activities or milestones that are centrally handled and occur prior to award letter or site selection; (b) “Central—2—Site Selected/Award Letter Received” which is a record type associated with trial activities or milestones that are centrally handled and occur after the award letter has been received but before the trial is running; (c) “Central—3—Trial Received—Running” which is a record type associated with trial activities or milestones of a centrally handled trial that is currently running at the sites; and (d) “Central—4—Trial Completed” which is a record type associated with a centrally handled trial that is now over and has been completed.

Examples of individually applicable record types include: (a) “Individual—1—Prior to Site Selection/Award Letter” which is a record type that is used when each site of the multiple sites is performing its own CDA, feasibility, contract, budget, and/or other trial-related. activities that occur before the trial is running; and (b) “Individual—2—Trial Received/Running” which is a record type that is used when each site is performing its own CDA, feasibility, contract, budget, and/or other trial-related activities that occur after the trial is running.

Example of record types that apply to the multiple sites as a whole include: (a) “Multi—Awarded but Passed” which is a record type associated with trials that are passed by the sites (e.g., for budgetary, contractual, or other issues); (b) “Multi—Historical Trial” which is a record type for recording historical data of trials that are already completed; (c) “Multi—Not Interested” which is a record type associated with trials that the site(s) were not interested in participating but nonetheless would like to track and capture performance or timing metrics on.

In one example embodiment, the clinical trial management platform 105 may create and program a proprietary roll-up feature that uses “look-up” relationships as opposed to Master-Detail relationships, thereby enabling unlimited number of “roll-up” calculations. In another embodiment, these rollups are created to perform these calculations and use advanced logic to calculate and display these data points based on formula fields. Further, these roll-up calculations of unique metrics that are important in the clinical research industry and the displaying of these metrics may be built into the Accounts, Indications, Contacts, Trials and Advertising sections.

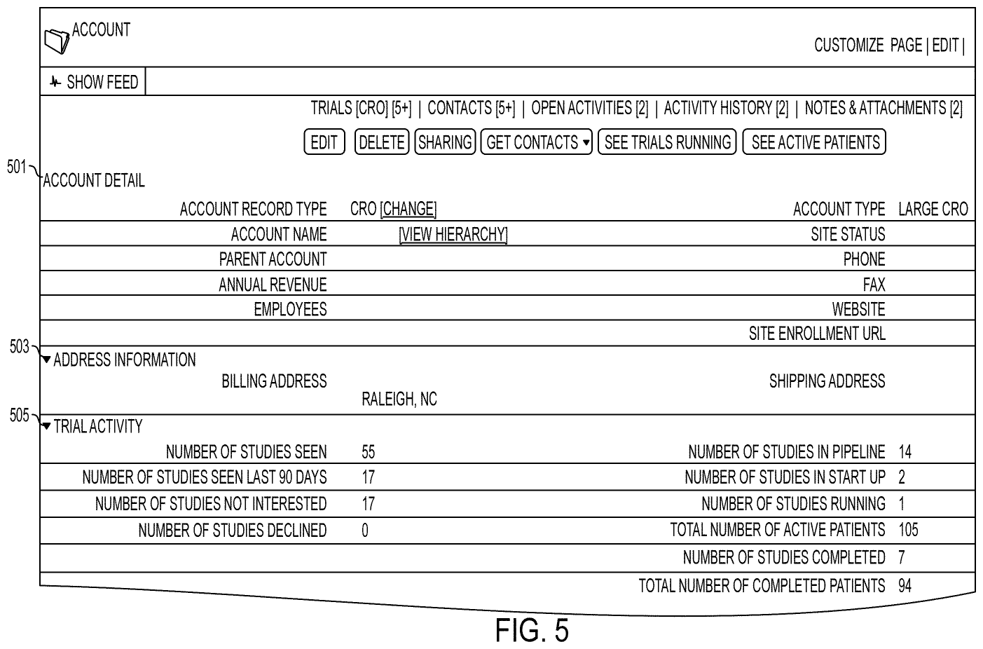

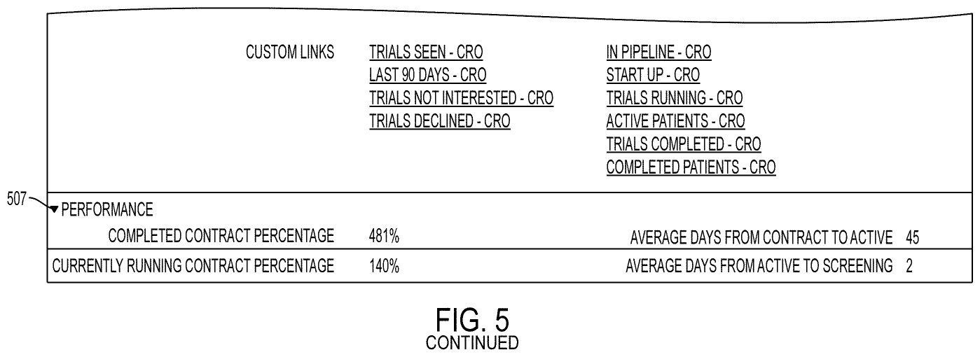

FIG. 5 is a diagram that represents Contract Research Organizations (CRO) rollups, according to one example embodiment. In one example embodiment, the CRO rollups includes account detail 501, address information 503, trial activity 505 and performance 507. In one scenario, the account detail 501 may comprise of account record type, account name, parent account, annual revenue, employees, account type, site status, phone number, fax information, website information, and site enrollment URL. In another scenario, the trial activity 505 may comprise of number of studies seen, number of studies seen last 90 days, number of studies not interested, number of studies declined, number of studies in pipeline, number of studies in startup, number of studies running, total number of active patients, number of studies completed, and total number of completed patients. In a further scenario, the performance 507 may comprise of completed contract percentage, currently running contract percentage, average days from active to screening, and average days from contract to active.

FIG. 6 is a diagram that represents sponsor rollups, according to one example embodiment. In one example embodiment, the sponsor rollups may include account detail 601 which may further include information on account record types, account name, parent account, annual revenue, employees, account type, site status, phone number, fax number, website, and site enrollment URL. In another example embodiment, the sponsor rollups may include address information 603, trial activity 605, and performance 607. In one scenario, the trial activity 605 may include but is not limited to information on number of studies seen, number of studies seen last 90 days, number of studies not interested, number of studies declined, number of studies in pipeline, number of studies in startup, number of studies running, total number of active patients, number of studies completed, and total number of completed patients. Although the example of FIG. 6 is presented in the context of a single site, it is contemplated that the example sponsor rollups can also be applicable to or otherwise depict information drawn from multiple sites (e.g., according to a multiple site use case as described with respect to FIG. 4B above).

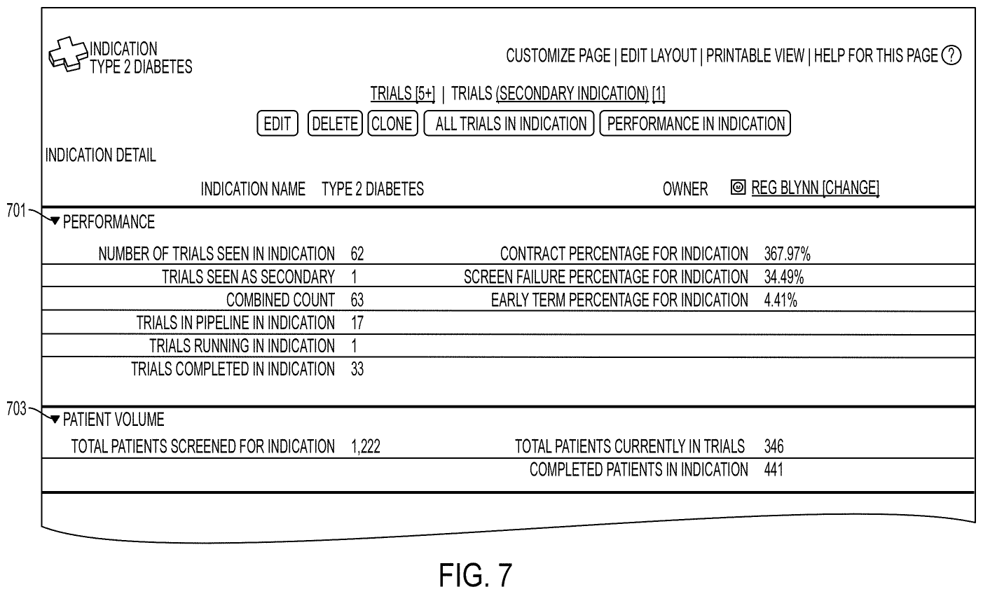

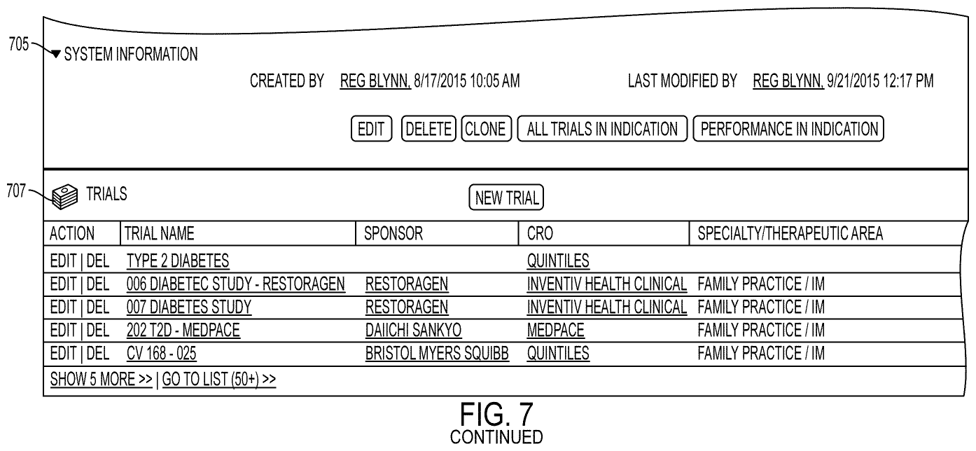

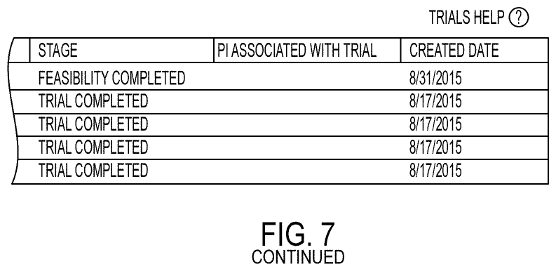

FIG. 7 is a diagram that represents indication rollups, according to one example embodiment. In one example embodiment, the indication rollups may include performance information 701, which may further include information on the number of trails seen in indication, trials seen as secondary, combined count, trials in pipeline in indication, trials running in indication, trials completed in indication, contract percentage for indication, screen failure percentage for indication, and early term percentage for indication. In another example embodiment, the indication rollups may include information on patient volume 703, for example, the total patients screened for indication, the total patients currently in trials, and completed patients in indication. In a further example embodiment, the indication rollups may include information on system information 705 and trials 707. In one scenario, trials 707 may include trial name, sponsor information, CRO information, specialty/therapeutic area, stage, PI associated with trail, etc.

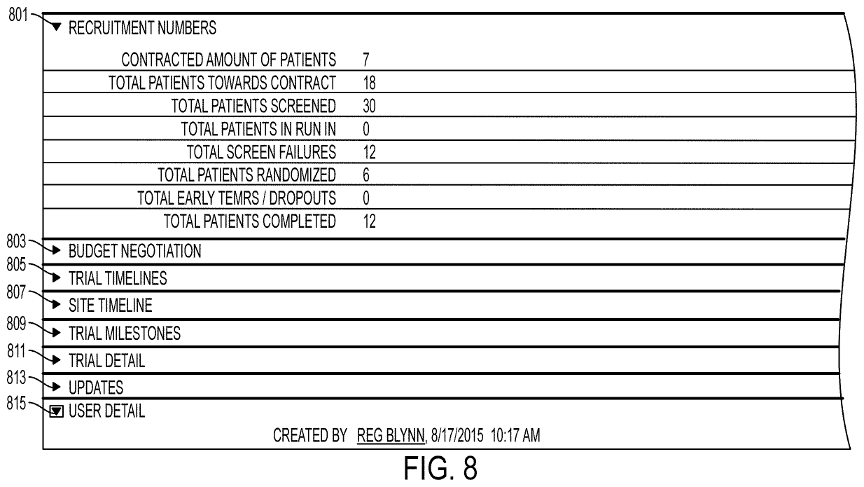

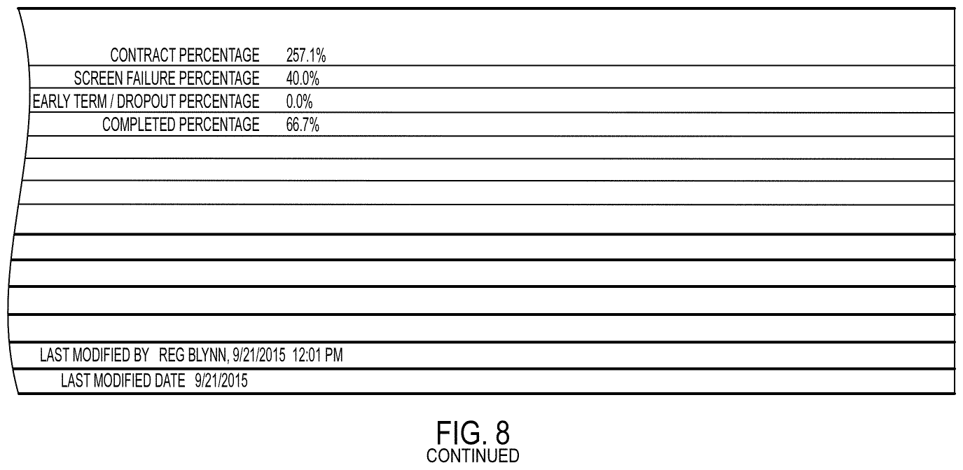

FIG. 8 is a diagram that represents enrollment update rollups, according to one example embodiment. In one example embodiment, the enrollment update rollups may comprise of recruitment numbers 801. In one scenario, the recruitment numbers may include contracted amount of patients, total patients towards contract, total patients screened, total patients in run in, total screen failures, total patients randomized, total early terms/dropouts, total patients completed, contract percentage, screen failure percentage, early term/dropout percentage, and completed percentage. In another example embodiment, the enrollment update rollups may further comprise information on budget negotiation 803, trial timelines 805, site timelines 807, trial milestones 809, trial detail 811, updates 813, and user details 815. In a further example embodiment, the enrollment update rollups includes trial contacts 817, which may include but is not limited to information on name of the contact, account name, role of the contact, status, trial contact, email information, and phone number.

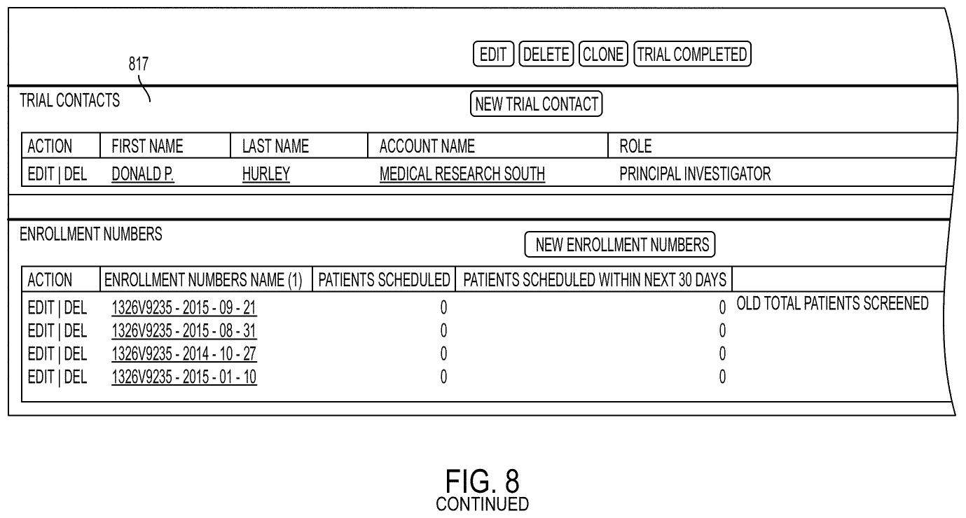

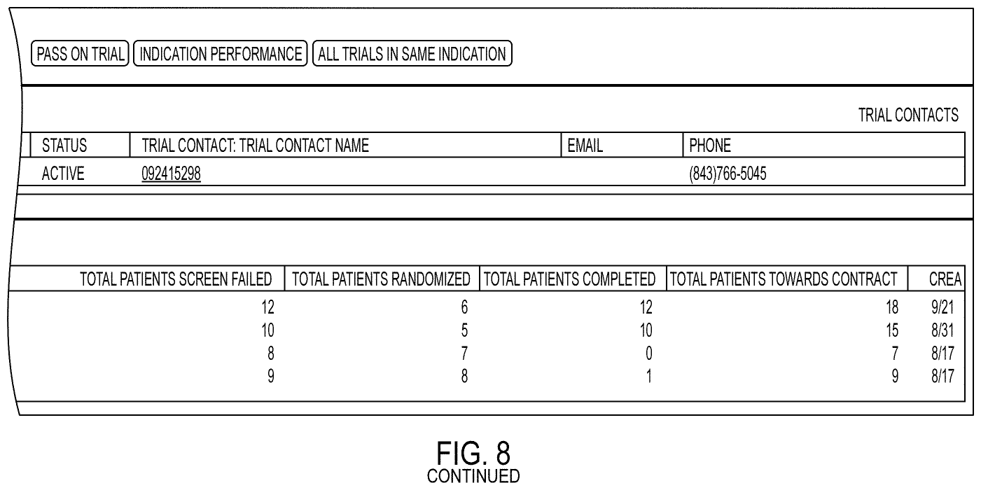

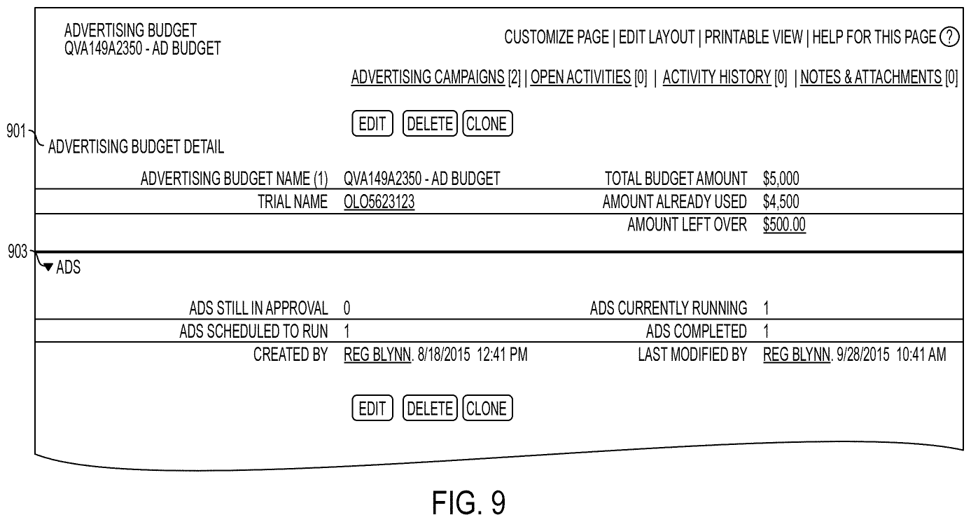

FIG. 9 is a diagram that represents advertisement campaigns rollups, according to one example embodiment. In one example embodiment, the advertisement campaign rollups may comprise of advertisement budget detail 901. In one scenario, the advertisement budget detail 903 may include advertisement budget name, total budget amount, trial name, amount used and the remaining amount. In another example embodiment, the advertisement campaign rollups may also include information on advertisement that is still in approval state, advertisement that is scheduled to run, advertisement that is currently running, advertisement that was complete, or a combination thereof. In a further example embodiment, the advertisement campaign rollups may also include information on advertising campaigns 905, which may further include but is not limited to information on advertising campaign name, advertising vendor, advertisement status, IRB status, sponsor status, and amount spent.

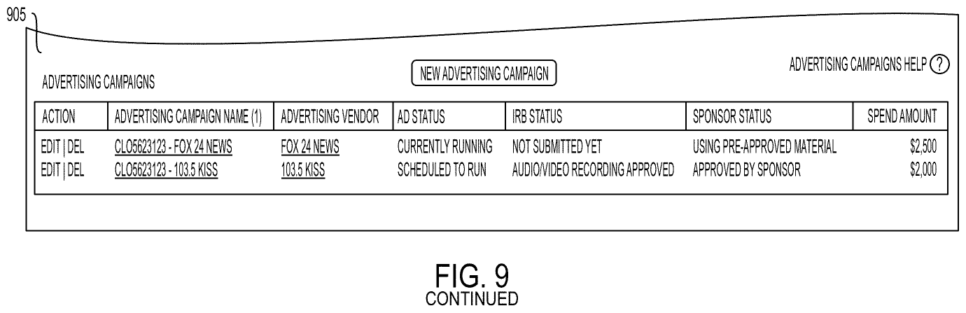

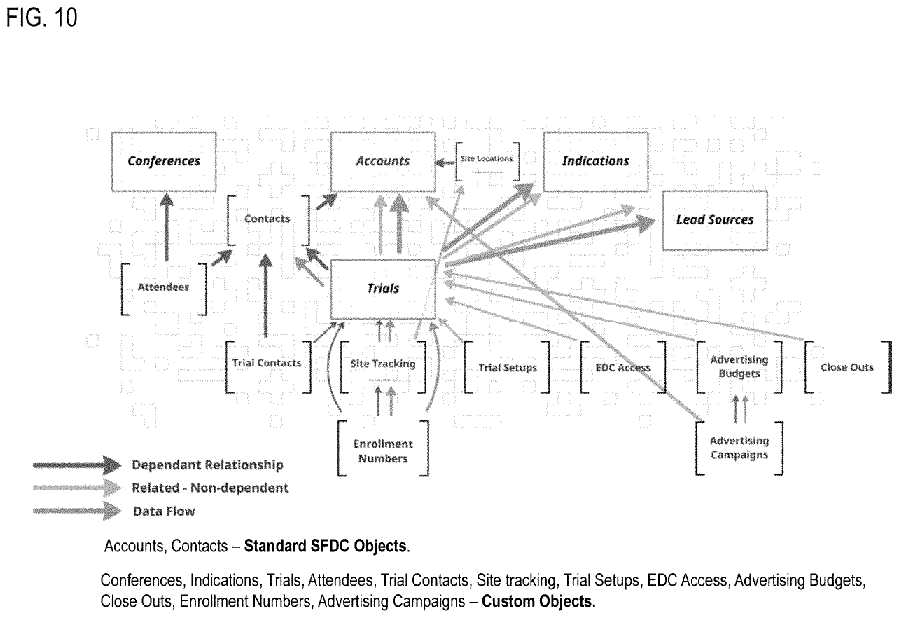

FIG. 10 is a flow chart that represents the relationship of all objects (e.g., both standard and custom) in, for instance, a Salesforce.com development environment, according to one example embodiment. In one embodiment, the clinical trial management platform 105 may incorporate a massive amount of custom objects, automated workflows and apex coding to allow data to flow across custom and standard objects in ways that SFDC (Salesforce Dot Com) would not normally allow. The SFDC only allows a limited amount of rollup fields based on Master-Detail relationships. The problem is there are objects that are not related to each other in a way where the Master-Detail relationship may work as they are not mandatory fields. Hence, the clinical trial management platform 105 may incorporate multiple (e.g., over 900 lines) Apex code to allow unlimited and non-relationship restricted rollups.

The processes described herein for managing clinical trials and research may be advantageously implemented via software, hardware (e.g., general processor, Digital Signal Processing (DSP) chip, an Application Specific Integrated Circuit (ASIC), Field Programmable Gate Arrays (FPGAs), etc.), firmware or a combination thereof. Such exemplary hardware for performing the described functions is detailed below.

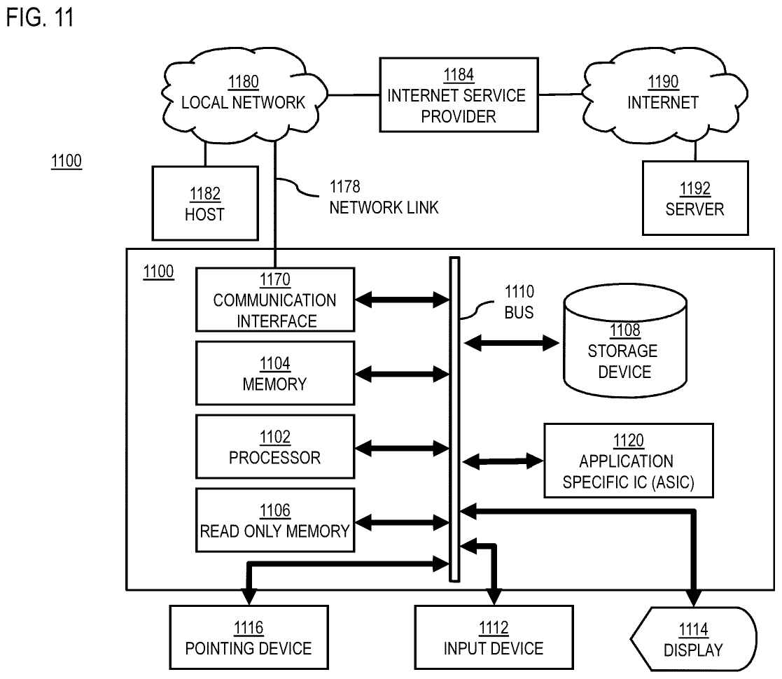

FIG. 11 illustrates a computer system 1100 upon which an embodiment of the invention may be implemented. Computer system 1100 is programmed (e.g., via computer program code or instructions) to manage clinical trials and research as described herein and includes a communication mechanism such as a bus 1110 for passing information between other internal and external components of the computer system 1100. Information (also called data) is represented as a physical expression of a measurable phenomenon, typically electric voltages, but including, in other embodiments, such phenomena as magnetic, electromagnetic, pressure, chemical, biological, molecular, atomic, sub-atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range.

A bus 1110 includes one or more parallel conductors of information so that information is transferred quickly among devices coupled to the bus 1110. One or more processors 1102 for processing information are coupled with the bus 1110.

A processor 1102 performs a set of operations on information as specified by computer program code related to managing clinical trials and research. The computer program code is a set of instructions or statements providing instructions for the operation of the processor and/or the computer system to perform specified functions. The code, for example, may be written in a computer programming language that is compiled into a native instruction set of the processor. The code may also be written directly using the native instruction set (e.g., machine language). The set of operations include bringing information in from the bus 1110 and placing information on the bus 1110. The set of operations also typically include comparing two or more units of information, shifting positions of units of information, and combining two or more units of information, such as by addition or multiplication or logical operations like OR, exclusive OR (XOR), and AND. Each operation of the set of operations that can be performed by the processor is represented to the processor by information called instructions, such as an operation code of one or more digits. A sequence of operations to be executed by the processor 1102, such as a sequence of operation codes, constitute processor instructions, also called computer system instructions or, simply, computer instructions. Processors may be implemented as mechanical, electrical, magnetic, optical, chemical or quantum components, among others, alone or in combination.

Computer system 1100 also includes a memory 1104 coupled to bus 1110. The memory 1104, such as a random access memory (RAM) or other dynamic storage device, stores information including processor instructions for managing clinical trials and research. Dynamic memory allows information stored therein to be changed by the computer system 1100. RAM allows a unit of information stored at a location called a memory address to be stored and retrieved independently of information at neighboring addresses. The memory 1104 is also used by the processor 1102 to store temporary values during execution of processor instructions. The computer system 1100 also includes a read only memory (ROM) 1106 or other static storage device coupled to the bus 1110 for storing static information, including instructions, that is not changed by the computer system 1100. Some memory is composed of volatile storage that loses the information stored thereon when power is lost. Also coupled to bus 1110 is a non-volatile (persistent) storage device 1108, such as a magnetic disk, optical disk or flash card, for storing information, including instructions, that persists even when the computer system 1100 is turned off or otherwise loses power.

Information, including instructions for managing clinical trials and research, is provided to the bus 1110 for use by the processor from an external input device 1112, such as a keyboard containing alphanumeric keys operated by a human user, or a sensor. A sensor detects conditions in its vicinity and transforms those detections into physical expression compatible with the measurable phenomenon used to represent information in computer system 1100. Other external devices coupled to bus 1110, used primarily for interacting with humans, include a display device 1114, such as a cathode ray tube (CRT) or a liquid crystal display (LCD), or plasma screen or printer for presenting text or images, and a pointing device 1116, such as a mouse or a trackball or cursor direction keys, or motion sensor, for controlling a position of a small cursor image presented on the display 1114 and issuing commands associated with graphical elements presented on the display 1114. In some embodiments, for example, in embodiments in which the computer system 1100 performs all functions automatically without human input, one or more of external input device 1112, display device 1114 and pointing device 1116 is omitted.

In the illustrated embodiment, special purpose hardware, such as an application specific integrated circuit (ASIC) 1120, is coupled to bus 1110. The special purpose hardware is configured to perform operations not performed by processor 1102 quickly enough for special purposes. Examples of application specific ICs include graphics accelerator cards for generating images for display 1114, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1100 also includes one or more instances of a communications interface 1170 coupled to bus 1110. Communication interface 1170 provides a one-way or two-way communication coupling to a variety of external devices that operate with their own processors, such as printers, scanners and external disks. In general the coupling is with a network link 1178 that is connected to a local network 1180 to which a variety of external devices with their own processors are connected. For example, communication interface 1170 may be a parallel port or a serial port or a universal serial bus (USB) port on a personal computer. In some embodiments, communications interface 1170 is an integrated services digital network (ISDN) card or a digital subscriber line (DSL) card or a telephone modem that provides an information communication connection to a corresponding type of telephone line. In some embodiments, a communication interface 1170 is a cable modem that converts signals on bus 1110 into signals for a communication connection over a coaxial cable or into optical signals for a communication connection over a fiber optic cable. As another example, communications interface 1170 may be a local area network (LAN) card to provide a data communication connection to a compatible LAN, such as Ethernet. Wireless links may also be implemented. For wireless links, the communications interface 1170 sends or receives or both sends and receives electrical, acoustic or electromagnetic signals, including infrared and optical signals, that carry information streams, such as digital data. For example, in wireless handheld devices, such as mobile telephones like cell phones, the communications interface 1170 includes a radio band electromagnetic transmitter and receiver called a radio transceiver. In certain embodiments, the communications interface 1170 enables connection to the communication network 103 for managing clinical trials and research.

The term computer-readable medium is used herein to refer to any medium that participates in providing information to processor 1102, including instructions for execution. Such a medium may take many forms, including, but not limited to, non-volatile media, volatile media and transmission media. Non-volatile media include, for example, optical or magnetic disks, such as storage device 1108. Volatile media include, for example, dynamic memory 1104. Transmission media include, for example, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization or other physical properties transmitted through the transmission media. Common forms of computer-readable media include, for example, a floppy disk, a flexible disk, hard disk, magnetic tape, any other magnetic medium, a CD-ROM, CDRW, DVD, any other optical medium, punch cards, paper tape, optical mark sheets, any other physical medium with patterns of holes or other optically recognizable indicia, a RAM, a PROM, an EPROM, a FLASH-EPROM, any other memory chip or cartridge, a carrier wave, or any other medium from which a computer can read.

FIG. 12 illustrates a chip set 1200 upon which an embodiment of the invention may be implemented. Chip set 1200 is programmed to manage clinical trials and research as described herein and includes, for instance, the processor and memory components described with respect to FIG. 11 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip.

In one embodiment, the chip set 1200 includes a communication mechanism such as a bus 1201 for passing information among the components of the chip set 1200. A processor 1203 has connectivity to the bus 1201 to execute instructions and process information stored in, for example, a memory 1205. The processor 1203 may include one or more processing cores with each core configured to perform independently. A multi-core processor enables multiprocessing within a single physical package. Examples of a multi-core processor include two, four, eight, or greater numbers of processing cores. Alternatively or in addition, the processor 1203 may include one or more microprocessors configured in tandem via the bus 1201 to enable independent execution of instructions, pipelining, and multithreading. The processor 1203 may also be accompanied with one or more specialized components to perform certain processing functions and tasks such as one or more digital signal processors (DSP) 1207, or one or more application-specific integrated circuits (ASIC) 1209. A DSP 1207 typically is configured to process real-world signals (e.g., sound) in real time independently of the processor 1203. Similarly, an ASIC 1209 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1203 and accompanying components have connectivity to the memory 1205 via the bus 1201. The memory 1205 includes both dynamic memory (e.g., RAM, magnetic disk, writable optical disk, etc.) and static memory (e.g., ROM, CD-ROM, etc.) for storing executable instructions that when executed perform the inventive steps described herein to managing clinical trials and research. The memory 1205 also stores the data associated with or generated by the execution of the inventive steps.

While the invention has been described in connection with a number of embodiments and implementations, the invention is not so limited but covers various obvious modifications and equivalent arrangements, which fall within the purview of the appended claims. Although features of the invention are expressed in certain combinations among the claims, it is contemplated that these features can be arranged in any combination and order.

1. (canceled)

2. A method of monitoring a plurality of clinical trial processes, comprising:
receiving, via a user interface, an input specifying modification of a data record associated with one of the clinical trial processes, wherein the data record includes a plurality of fields corresponding respectively to a plurality of stages of the one clinical trial process;
determining a particular stage among the plurality of stages corresponding to the data record;
presenting, via the user interface, one or more user interface elements corresponding to the particular stage;
assigning a workflow rule to an action based on the one or more user interface elements;
evaluating the input according to the workflow rule to initiate the action;
determining a performance metric for the action, wherein the performance metric specifies timing information and cost information for the particular stage;
updating the data record based on the performance metric; and
modifying the one or more user interface elements of the user interface according to the updated data record.

3. The method of claim 2, further comprising:
determining a plurality of other performance metrices associated with other ones of the plurality of clinical trial processes; and
computing a normalization of the performance metric based on the plurality of other performance metrices, wherein the data record is updated based on the normalized performance metric.

4. The method of claim 2, further comprising:
determining a timing metric for the data record, wherein the timing metric specifies timing information for completion of the particular stage, wherein the data record is further updated based timing metric.

5. The method of claim 4, further comprising:
determining availability of the one or more user interface elements based on type of the data record, wherein the user interface elements comprise menus, buttons, data entry fields, or a combination thereof.

6. The method of claim 2, wherein the data record is dynamically updated according to real-time determination of the performance metric.

7. The method of claim 2, wherein the performance metric includes a cost metric, the method further comprising:
determining the cost metric for completing the particular stage, wherein the cost metric include a budget information for the one clinical trial process.

8. The method of claim 2, further comprising:
calculating a date for completing a subsequent stage of the one clinical trial process based on historical timing metrics, historical performance metrices, or a combination thereof and presenting, via the user interface, a notification associated with the calculated date.

9. An apparatus for monitoring a plurality of clinical trial processes, comprising:
at least one processor; and
at least one memory including computer program code for one or more programs,
the at least one memory and the computer program code configured to, with the at least one processor, cause the apparatus to perform at least the following,
receive, via a user interface, an input specifying modification of a data record associated with one of the clinical trial processes, wherein the data record includes a plurality of fields corresponding respectively to a plurality of stages of the one clinical trial process;
determine a particular stage among the plurality of stages corresponding to the data record;
present, via the user interface, one or more user interface elements corresponding to the particular stage;
assign a workflow rule to an action based on the one or more user interface elements;
evaluate the input according to the workflow rule to initiate the action;
determine a performance metric for the action, wherein the performance metric specifies timing information and cost information for the particular stage;
update the data record based on the performance metric; and
modify the one or more user interface elements of the user interface according to the updated data record.

10. The apparatus of claim 9, wherein the apparatus is further caused to:
determine a plurality of other performance metrices associated with other ones of the plurality of clinical trial processes; and
compute a normalization of the performance metric based on the plurality of other performance metrices, wherein the data record is updated based on the normalized performance metric.

11. The apparatus of claim 9, wherein the apparatus is further caused to:
determine a timing metric for the data record, wherein the timing metric specifies timing information for completion of the particular stage, wherein the data record is further updated based timing metric.

12. The apparatus of claim 11, wherein the apparatus is further caused to:
determine availability of the one or more user interface elements based on type of the data record, wherein the user interface elements comprise menus, buttons, data entry fields, or a combination thereof.

13. The apparatus of claim 9, wherein the data record is dynamically updated according to real-time determination of the performance metric.

14. The apparatus of claim 9, wherein the performance metric includes a cost metric, the apparatus being further caused to:
determine the cost metric for completing the particular stage, wherein the cost metric include a budget information for the one clinical trial process.

15. The apparatus of claim 9, wherein the apparatus is further caused to:
calculate a date for completing a subsequent stage of the one clinical trial process based on historical timing metrics, historical performance metrices, or a combination thereof; and
present, via the user interface, a notification associated with the calculated date.

16. A system comprising:
one or more servers are caused to perform at least the following,
initiate establishment of a communication session over a data network with a first clinical trial site;
receive, via a user interface, an input specifying modification of a data record associated with a clinical trial process maintained by the first clinical trial site, wherein the data record includes a plurality of fields corresponding respectively to a plurality of stages of the clinical trial process;
determine a particular stage among the plurality of stages corresponding to the data record;
present, via the user interface, one or more user interface elements corresponding to the particular stage;
assign a workflow rule to an action based on the one or more user interface elements;
evaluate the input according to the workflow rule to initiate the action;
determine a performance metric for the action, wherein the performance metric specifies timing information and cost information for the particular stage;
update the data record based on the performance metric;
modify the one or more user interface elements of the user interface according to the updated data record;
initiate storage of the updated data record for the first clinical trial site;
establish another communication session with a second clinical trial site; and
presenting, via the user interface, a plurality of centrally handled record types for the first clinical trial site and the second clinical trial site.

17. The system of claim 16, wherein the plurality of centrally handled record types are presented with a corresponding plurality of sections of the user interface, the plurality of centrally handled record types include:
a first type relating to data records about the first trial site and the second trial site before being selected;
a second type relating to data records about selection and award of the first trial site and the second trial site;
a third type relating to data records about activities associated with trials being running at the first trial site and the second trial site; and
a fourth type relating to data records about completed trials at the first trial site and the second trial site.

18. The system of claim 16, wherein the one or more servers are further caused to perform the following:
determine trial activity information about a plurality of clinical trials running at first trial site and the second trial site, wherein the trial activity information represents an aggregation of information for the first trial site and the second trial site; and
present, via the user interface, the trial activity information.

19. The system of claim 18, wherein the trial activity information relates to number of studies seen, number of studies seen during a particular period, number of studies not interested, number of studies declined, number of studies in pipeline, number of studies in startup, number of studies running, total number of active patients, number of studies completed, total number of completed patients, or a combination thereof.

20. The system of claim 18, wherein the trial activity information include sponsorship information, contract research organization information, advertisement campaign information, or a combination thereof.

21. The system of claim 16, wherein the data record is dynamically updated according to real-time determination of the performance metric.
